(19) United States
(12) Patent Application Publication
(10) Pub. No.: US 2005/0204416 A1
(43) Pub. Date: Sep. 15, 2005

(54) Plant cells having receptor polypeptides

(76) Inventors: Richard Hamilton, Calabasas, CA (US); Nestor Apuya, Culver City, CA (US); Steven Craig Bobzin, Malibu, CA (US)

Correspondence Address: FISH & RICHARDSON P.C., PO BOX 1022, MINNEAPOLIS, MN, 55440-1022, US

(21) Appl. No.: 11/035,623

(22) Filed: Jan. 14, 2005

Related U.S. Application Data
(60) Provisional application No. 60/537,070, filed on Jan. 16, 2004.

(51) Int. Cl. C12Q 1/00; C12Q 1/68; A01H 1/00; C12N 15/82; C12N 5/04

(52) U.S. Cl. 800/278; 435/4; 435/6; 435/419; 435/468

(57) ABSTRACT

Disclosed are novel transgenic plant cells that include a heterologous (e.g., human) receptor polypeptide, or fragment thereof. The plant cells can be used to identify molecules (i.e., ligands) that can interact with the receptor polypeptide or fragment. The plant cells can be used to identify ligands that are endogenous to transgenic plant cells, or exogenous ligands that are applied to the plant cells. Such receptor polypeptide ligands can be used to identify novel pharmaceuticals.

### CROSS-REFERENCE TO RELATED APPLICATIONS

This application claims priority from U.S. Provisional Application Ser. No. 60/537,070, filed Jan. 16, 2004, under 35 U.S.C. §119(e).

### TECHNICAL FIELD

This invention relates to methods and materials useful for identifying molecules that can interact with receptor polypeptides. In particular, the invention relates to methods utilizing transgenic plant cells to identify ligands that interact with nuclear hormone receptor polypeptides.

### BACKGROUND

Historically, plants have been a rich source of chemicals, which have been proven to be potent drugs. One example is the wild Mexican yarn, the inedible “cabeza de negro” yarn root growing in the mountains of Veracruz. This plant produces a steroid that can be transformed into cortisone or into the female sex hormone, progesterone, a precursor of estradiol.

Many clinically active drugs interact with receptor polypeptides, acting either as agonists or as antagonists of a receptor's cognate signaling molecules. Examples of such drugs derived from plants are listed in the Table below.

| Drug/Chemical Name | Source | Medical Condition |
|---|---|---|
| Deserpine | Rauwolfia canescens | Antihypertensive, |
| | | tranquillizer |
| Oxycodone (Percodan) | Papaver somniferum | Analgesic |
| Yohimbine | Pausinystalia yohimbe, | Aphrodisiac, |
| | Rauwolfia serpentina | antidepressant |

Assays to identify compounds that act as agonists or antagonists of receptors are desirable. See, e.g., U.S. Pat. No. 5,665,543. However, many current assays to identify plant-derived compounds that act as agonists or antagonists of receptors are not capable of screening large numbers of compounds. Moreover, even when a plant-derived compound is identified as an agonist or antagonist of a receptor, many such compounds are found to have significant side effects when administered to mammals. There is a need to rapidly and efficiently identify more novel plant-derived compounds that are ligands of receptor polypeptides.

### SUMMARY

The invention features transgenic plant cells and methods for identifying ligands that can interact with receptor polypeptides (“receptors”) in plant cells transformed with a nucleic acid encoding a heterologous receptor polypeptide. Thus, the invention features a method for identifying a ligand for a receptor, which comprises providing a plurality of plant cells comprising a nucleic acid encoding a heterologous receptor polypeptide and determining whether one or more candidate ligands interact with the receptor, using a reporter responsive to signal transduction activity of the receptor. The plant cells can be a part of at least one whole plant, e.g., leaf cells or trichome cells. The plant cells can be cells in tissue culture. The plant cells can have been exposed to mechanical stress, thermal stress, or fungal stress.

The heterologous receptor can comprise a ligand binding domain, a DNA binding domain, and a transactivation domain. The heterologous receptor can be a nuclear hormone receptor polypeptide. A nuclear hormone receptor can be an insect nuclear hormone receptor, or can be a mammalian nuclear hormone receptor, e.g., AR, RAR, ROR, LRH-1, THR, VDR, PPAR, LXR, ER, CAR, FXR, or RXR. The plurality of plant cells can further comprise a nucleic acid encoding a dimerization receptor polypeptide. The heterologous receptor can be a chimeric receptor, e.g., a chimeric receptor that comprises a ligand binding domain, a DNA binding domain, and a transactivation domain. The transactivation domain of a chimeric receptor can be a VP 16 transactivation domain or a maize transcription factor C transactivation domain.

The DNA binding domain of a chimeric receptor can be a DNA binding domain of AR, RAR, ROR, LRH-1, THR, VDR, PPAR, LXR, ER, CAR, FXR, or RXR. A heterologous receptor can further include a dimerization sequence. A heterologous receptor can further include a localization signal. The localization signal can be a cytoplasmic localization signal, a nuclear localization signal, an ER localization signal, a Golgi apparatus localization signal, or a cell membrane localization signal.

The one or more candidate ligands can be synthesized by plant cells that are part of a whole plant. The candidate ligands can be endogenous ligands that are synthesized by the plurality of plant cells.

The reporter can be a polypeptide having a spectrophotometrically measurable activity, e.g., fluorescence or bioluminescence. The reporter polypeptide can comprise an epitope tag. The epitope tag can be a FLAG® tag, HA tag, c-Myc epitope, AU1 tag, or a 6-HIS tag. A reporter polypeptide can be GFP, GUS, YFP, RFP, luciferase or beta-galactosidase. The reporter polypeptide can be encoded by a recombinant nucleic acid in the plurality of plant cells and transcription of the recombinant nucleic acid can be mediated by the signal transduction activity of the receptor. The reporter can be responsive to a receptor response element capable of interacting with a DNA binding domain present in the heterologous receptor polypeptide.

The nucleic acid encoding a heterologous receptor polypeptide can be operably linked to a regulatory element conferring preferential expression in a plant tissue or organ such as a leaf, a root, a stem and a seed. In some embodiments, the nucleic acid encoding a heterologous receptor polypeptide can be operably linked to a regulatory element that confers constitutive expression.

The receptor polypeptide can comprise an epitope. The epitope can be naturally occurring or can be synthetic epitope such as a FLAG® or His tag. The method can further comprising using mass spectroscopy to identify the ligand.

The receptor polypeptide can comprise a signal sequence that targets the receptor to a membrane, e.g., selected from the group consisting of endoplasmic reticulum membrane, Golgi membrane, nuclear membrane, peroxisome membrane, mitochondrial membrane, chloroplast membrane, and plasma membrane.

The method can involve isolating cells that express the receptor, or using mass spectroscopy to identify the ligand. In some embodiments, the method involves isolating cells that express a reporter, or using mass spectroscopy to identify the ligand.

The plurality of plant cells can further comprise a nucleic acid encoding a sterol biosynthesis polypeptide, e.g., a squalene synthase polypeptide, a lupeol synthase polypeptide, a cycloartenol synthase polypeptide, a sterol methyl oxidase polypeptide, a diterpene synthase polypeptide, a sesquiterpene synthase polypeptide, or a terpenoid synthase polypeptide. The plurality of plant cells can further comprise a nucleic acid encoding a flavonoid biosynthesis polypeptide, e.g., a chalcone isomerase polypeptide, a chalcone reductase polypeptide, a dihydroflavonol reductase polypeptide, a isoflavone synthase polypeptide, a isoflavone reductase polypeptide, or a flavonoid 3-hydroxylase polypeptide. The plurality of plant cells can further contain a nucleic acid encoding a nucleic acid encoding a phenolic biosynthesis polypeptide, a terpenoid biosynthesis polypeptide, or an alkaloid biosynthesis polypeptide.

The invention also features a method of screening for a nuclear hormone receptor ligand. The method comprises providing one or more plant cells comprising at least one construct comprising a coding sequence for a nuclear hormone receptor polypeptide and a sequence to be transcribed as a reporter. The nuclear hormone receptor polypeptide comprises a ligand binding domain, a DNA binding domain, and a transactivation domain, wherein the sequence to be transcribed as a reporter is operably linked to a receptor response element capable of interacting with the DNA binding domain, and wherein activity of the reporter is detectable upon receptor/ligand binding-dependent transcription of the sequence. A candidate ligand is permitted to contact the receptor polypeptide under conditions that allow transcription of the receptor polypeptide from the construct and binding of the ligand thereto, and it is determined whether activity of the reporter is detected. The plant cells can be a part of at least one whole plant, e.g., leaf cells or trichome cells. The plant cells can be cells in tissue culture, e.g., a callus culture. The plant cells can have been exposed to mechanical stress, thermal stress, or fungal stress. The nuclear hormone receptor can be a mammalian nuclear hormone receptor, e.g., AR, RAR, ROR, LRH-1, THR, VDR, PPAR, LXR, ER, CAR, FXR, or RXR. The plurality of plant cells can further comprise a nucleic acid encoding a dimerization receptor polypeptide.

The nuclear hormone receptor can be a chimeric receptor. The transactivation domain of the chimeric receptor can be a VP16 transactivation domain or a maize transcription factor C transactivation domain. The DNA binding domain of the chimeric receptor can be a DNA binding domain of AR, RAR, ROR, LRH-1, THR, VDR, PPAR, LXR, ER, CAR, FXR, or RXR. The nuclear hormone receptor can further comprise a dimerization sequence. The coding sequence for a nuclear hormone receptor polypeptide can be operably linked to a regulatory element conferring preferential expression in a plant tissue or organ such as a leaf, a root, a stem and a seed. In some embodiments, the coding sequence for a nuclear hormone receptor polypeptide can be operably linked to a regulatory element that confers constitutive expression.

The nuclear hormone receptor can further comprise a localization signal, e.g., a cytoplasmic localization signal, a nuclear localization signal, an ER localization signal, a Golgi apparatus localization signal, or a cell membrane localization signal.

The one or more candidate ligands can be synthesized by plant cells, and the plant cells can be part or all of a whole plant. The candidate ligands can be endogenous ligands that are synthesized by the plurality of plant cells. The sequence to be transcribed can encode a polypeptide having a spectrophotometrically measurable activity, e.g., fluorescence or bioluminescence. The sequence to be transcribed can further encode an epitope tag. The epitope tag can be a FLAG® tag, HA tag, c-Myc epitope, AU1 tag, or a 6-HIS tag. The reporter polypeptide can be GFP, GUS, YFP, RFP, luciferase and beta-galactosidase.

The receptor polypeptide can comprise a signal sequence that targets the receptor to a membrane, e.g., endoplasmic reticulum membrane, Golgi membrane, nuclear membrane, peroxisome membrane, mitochondrial membrane, chloroplast membrane, or plasma membrane. The method can further comprise isolating cells that express the receptor. The method can further comprise using mass spectroscopy to identify the ligand.

The one or more plant cells can further comprise a nucleic acid encoding a sterol biosynthesis polypeptide, e.g., a squalene synthase polypeptide, a lupeol synthase polypeptide, a cycloartenol synthase polypeptide, or a sterol methyl oxidase polypeptide. The plurality of plant cells can further comprise a nucleic acid encoding a flavonoid biosynthesis polypeptide, e.g., a chalcone isomerase polypeptide, a chalcone reductase polypeptide, a dihydroflavonol reductase polypeptide, a isoflavone synthase polypeptide, a isoflavone reductase polypeptide, or a flavonoid 3-hydroxylase polypeptide. The plurality of plant cells can further contain a nucleic acid encoding a phenolic biosynthesis polypeptide, a terpenoid biosynthesis polypeptide, or an alkaloid biosynthesis polypeptide.

The invention also features a transgenic plant cell that contains a first recombinant nucleic acid encoding a polypeptide having greater than 40% sequence identity to a nuclear hormone receptor polypeptide, where the polypeptide is operably linked to a regulatory element, and a second recombinant nucleic acid that includes a nuclear receptor response element operably linked to a reporter coding sequence. The nuclear hormone receptor polypeptide can be AR, RAR, ROR, LRH-1, THR, VDR, PPAR, LXR, ER, CAR, FXR, or RXR. In some embodiments, the transgenic plant cell is a transiently transformed cell.

Other features and advantages of the invention will be apparent from the following detailed description, and from the claims. Unless otherwise defined, all technical and scientific terms used herein have the meaning commonly understood by one of ordinary skill in the art to which this invention belongs. All publications, patent applications, patents, and other references mentioned herein are incorporated by reference in their entirety. In case of conflict, the present specification, including definitions, will control. The disclosed materials, methods, and examples are illustrative only and not intended to be limiting. Skilled artisans will appreciate that methods and materials similar or equivalent to those described herein can be used to practice the invention.

### DESCRIPTION OF DRAWINGS

FIG. 1 shows the nucleotide sequence encoding a VP16ERα chimeric polypeptide.

FIG. 2 shows the coding sequence for a green fluorescent protein operably linked to five copies of a human estrogen response element. Underlined (straight line) nucleotides indicate the five copies of the estrogen response element. Underlined (wavy line) nucleotides indicate the minimal TATA sequence. Nucleotides in green font correspond to the coding sequence for the chimeric green fluorescent protein (GFP).

### DETAILED DESCRIPTION

The invention provides methods and materials for identifying molecules that interact with receptor polypeptides. In general, such molecules are referred to as ligands and can act as agonists or antagonists of a cognate signaling molecule of a receptor. The featured plants and techniques can be used to screen a wide variety of exogenous candidate ligands that are applied to transgenic plant cells, and to efficiently identify ligands that are endogenous to the transgenic plant cells. Ligands identified according to the featured methods may be pharmaceutically useful, acting as agonists or antagonists of a receptors' cognate signaling molecules.

Methods in accord with the invention involve the use of plant cells comprising a nucleic acid encoding a heterologous receptor to identify one or more candidate ligands that interact with the receptor, wherein the system also utilizes a reporter whose activity is responsive to modulation by the ligand of signal transduction activity of the heterologous receptor. In some embodiments, candidate ligands are present in the plant cells that express the heterologous receptor, thus providing a convenient, self-contained system for determining whether such plant cells possess ligands that interact with the receptor. Molecules present in plant cells exhibiting modulation of receptor activity can then be characterized in order to identify ligands that have not been identified heretofore by conventional methods.

I. Heterologous Receptors

Transgenic plants and plant cells for use in the methods described herein contain a nucleic acid encoding a heterologous receptor polypeptide. A heterologous receptor polypeptide is a receptor polypeptide that is not present in non-transgenic counterparts to plant cells to be used in the method. A heterologous receptor polypeptide can be the polypeptide encoded by a full-length coding sequence of a naturally occurring receptor. A number of heterologous receptor polypeptides are suitable for use in the methods described herein.

Of particular interest are nuclear hormone receptors, particularly human nuclear hormone receptors. Nuclear hormone receptors are a large family of gene regulatory, DNA-binding proteins that bind hormonally and nutritionally derived lipophilic ligands. Nuclear hormone receptors have long been known to be DNA-binding proteins that can activate or repress transcription of target genes. Many nuclear hormone receptors have been identified, including, for example, retinoid X receptor, retinoic acid receptor, progesterone receptor, estrogen receptor, androgen receptor, and vitamin D receptor. Nuclear hormone receptors are thought to have been conserved throughout evolution and to play a role in cell growth and proliferation, development and homeostasis. Changes in nuclear hormone receptors have been implicated in a number of diseases.

In particular embodiments, a heterologous receptor polypeptide present in plant cells can be: retinoid X receptor (RXR), hepatocyte nuclear factor 4 (HFN4), testicular receptor, tailless gene homolog (TLX), chicken ovalbumin upstream promoter transcription factor (COUP-TF), thyroid hormone receptor (THR), retinoic acid receptor (RAR), peroxisome proliferator activated receptor (PPAR), reverse Erb (reverb), RAR-related orphan receptor (ROR), Steroidogenic factor-1 (SF-1), liver receptor homolog-1 (LRH-1), liver X receptor (LXR), famesoid X receptor (FXR), vitamin D receptor (VDR), ecdysone receptor (EcR), pregnane X receptor (PXR), constitutive androstane receptor (CAR), neuron-derived activated receptor (NOR1), nuclear receptor 1 (NURR1), estrogen receptor (ER), estrogen-related receptor (ERR), glucocorticoid receptor (GR), androgen receptor (AR), progesterone receptor (PR), or mineralocorticoid receptor (MR).

An exemplary nuclear hormone receptor is a PPAR receptor polypeptide. PPAR receptors are considered to be members of a superfamily of nuclear hormone receptors. PPAR is activated upon binding of a ligand, and binding of PPAR/ligand in the form of a heterodimer to a response sequence (peroxisome proliferator response element, PPRE) activates transcription of a sequence operably positioned downstream of the PPRE. PPAR receptor polypeptides have been categorized into three subtypes called PPARα, PPARβ and PPARγ, and cDNA sequences obtained. See, e.g., U.S. patent publication 20020119499.

Another exemplary nuclear hormone receptor is an ER polypeptide. Estrogen receptors are activated upon binding of a ligand such as estrogen. Binding of an ER homodimer/ligand complex to a cis-responsive estrogen receptor element (ERE) activates transcription of a sequence operably positioned 3′ to the ERE. Two estrogen receptors are known in humans, ERα and ERβ. The two receptors share common structural and functional domains: they bind to estrogen with high affinity, and bind estrogen response elements in a similar manner, but they differ in with respect to tissue distribution, transcriptional activities, and phenotypes in knockout models.

A retinoid X receptor (RXR) can bind DNA as a homodimer in a ligand dependent manner at a RXR response element. One such ligand is 9-cis retinoic acid. RXR can also form a functional heterodimer with retinoic acid receptor (RAR), thyroid hormone receptor, vitamin D receptor, NGFI-B and other nuclear receptors. In mouse, retinoid X receptors have been categorized into three subtypes, designated RXRα (RXRA), RXRβ (RXRB), and RXRγ (RXRG).

A hepatocyte nuclear factor 4 (HFN4) can bind DNA as a homodimer in a ligand dependent manner at a response element comprised of two core motifs, 5′-RG(G/T)TCA, or a closely related sequence separated by 1 nucleotide (direct repeat, or DR1 elements).

A testicular receptor 2 (TR2) can bind DNA as a homodimer in a ligand dependent manner. A TR2/ligand complex binds to a response element comprising two AGGTCA half-site direct repeat sequences with various spacings. Such a complex also can bind to response elements such as cellular retinol-binding protein II promoter region (CRBPIIp), SV40+55 region, and retinoic acid response element beta (RARE beta).

Thyroid hormone receptor polypeptides (THR) can bind DNA as homodimers and heterodimers (e.g., with retinoid X receptor). These polypeptides can bind a THR response element. Unlike steroid hormone receptors, thyroid hormone receptors can bind DNA in the absence of a ligand, which can result in decrease in transcription. Upon hormone binding, the receptor changes conformation which causes it to exert a positive effect on transcription. In humans, thyroid hormone receptors have been categorized into two subtypes, designated THRα (THRA) and THRβ (THRB).

Retinoic Acid receptor (RARs) polypeptides can bind DNA as homodimers in a ligand dependent manner at a RAR element (RARE). One such ligand is retinoic acid. Several RAR isoforms are known in mammals, including RARα, RARβ and RARγ.

Androgen receptor (AR) can bind DNA as a homodimer in a ligand dependent manner to an androgen response element (ARE). One such ligand is 7 alpha-methyl-17 alpha-(2′-(E)-iodovinyl)-19-nortestosterone.

Suitable heterologous receptor polypeptides can be identified in a variety of ways. Coimmunoprecipitation assays using antibodies against known receptors can be used to identify candidate polypeptides. Another way to identify candidate polypeptides is by functional complementation of receptor mutants. Suitable candidates for receptors also can be identified by analysis of nucleotide and polypeptide sequence alignments. For example, performing a query on a database of nucleotide or polypeptide sequences can identify orthologs of heterologous receptor polypeptides. Sequence analysis can involve BLAST or PSI-BLAST analysis of nonredundant databases using known receptor amino acid sequences. Those proteins in the database that have greater than 40% sequence identity are candidates for further evaluation for suitability as a receptor. If desired, manual inspection of such candidates can be carried out in order to narrow the number of candidates to be further evaluated. Manual inspection can be performed by selecting those candidates that appear to have domains suspected of being present in receptors.

A percent identity for any subject nucleic acid or amino acid sequence, e.g., a human ERα receptor polypeptide, relative to another “target” nucleic acid or amino acid sequence can be determined as follows. First, a target nucleic acid or amino acid sequence can be compared and aligned to a subject nucleic acid or amino acid sequence, using the BLAST 2 Sequences (Bl2seq) program from the stand-alone version of BLASTZ containing BLASTN and BLASTP (e.g., version 2.0.14). The stand-alone version of BLASTZ can be obtained at <www.fr.com/blast> or www.ncbi.nlm.nih.gov>. Instructions explaining how to use BLASTZ, and specifically the Bl2seq program, can be found in the ‘readme’ file accompanying BLASTZ. The programs also are described in detail by Karlin et al, 1990, Proc. Natl. Acad. Sci. 87:2264; Karlin et al, 1990, Proc. Natl. Acad. Sci. 90:5873; and Altschul et al, 1997, Nucl. Acids Res. 25:3389.

Bl2seq performs a comparison between the subject sequence and a target sequence using either the BLASTN (used to compare nucleic acid sequences) or BLASTP (used to compare amino acid sequences) algorithm. Typically, the default parameters of a BLOSUM62 scoring matrix, gap existence cost of 11 and extension cost of 1, a word size of 3, an expect value of 10, a per residue cost of 1 and a lambda ratio of 0.85 are used when performing amino acid sequence alignments. The output file contains aligned regions of homology between the target sequence and the subject sequence. Once aligned, a length is determined by counting the number of consecutive nucleotides or amino acid residues (i.e., excluding gaps) from the target sequence that align with sequence from the subject sequence starting with any matched position and ending with any other matched position. A matched position is any position where an identical nucleotide or amino acid residue is present in both the target and subject sequence. Gaps of one or more residues can be inserted into a target or subject sequence to maximize sequence alignments between structurally conserved domains (e.g., α-helices, β-sheets, and loops).

The percent identity over a particular length is determined by counting the number of matched positions over that particular length, dividing that number by the length and multiplying the resulting value by 100. For example, if (i) a 500 amino acid target sequence is compared to a subject amino acid sequence, (ii) the Bl2seq program presents 200 amino acids from the target sequence aligned with a region of the subject sequence where the first and last amino acids of that 200 amino acid region are matches, and (iii) the number of matches over those 200 aligned amino acids is 180, then the 500 amino acid target sequence contains a length of 200 and a sequence identity over that length of 90% (i.e., 180÷200×100=90). In some embodiments, the amino acid sequence of a suitable heterologous receptor polypeptide has greater than 40% sequence identity (e.g., >80%, >70%, >60%, >50% or >40%) to the amino acid sequence of human ERα polypeptide. In other embodiments, the amino acid sequence of a suitable heterologous receptor polypeptide has greater than 40% sequence identity (e.g., >80%, >70%, >60%, >50% or >40%) to the amino acid sequence of the human PPARα polypeptide. In certain other embodiments, the amino acid sequence of a suitable heterologous receptor polypeptide has greater than 40% sequence identity (e.g., >80%, >70%, >60%, >50% or >40%) to the amino acid sequence of retinoid X receptor (RXR) polypeptide, hepatocyte nuclear factor 4 (HFN4) polypeptide, testicular receptor polypeptide, tailless gene homolog (TLX) polypeptide, chicken ovalbumin upstream promoter transcription factor (COUP-TF) polypeptide, thyroid hormone receptor-α (THRA) polypeptide, thyroid hormone receptor-β (THRB) polypeptide, retinoic acid receptor (RAR) polypeptide, peroxisome proliferator activated receptor (PPAR) polypeptide, reverse Erb (reverb) polypeptide, RAR-related orphan receptor (ROR) polypeptide, Steroidogenic factor-1 (SF-1) polypeptide, liver receptor homolog-1 (LRH-1) polypeptide, liver X receptor (LXR) polypeptide, farnesoid X receptor (FXR) polypeptide, vitamin D receptor (VDR) polypeptide, ecdysone receptor (EcR) polypeptide, pregnane X receptor (PXR) polypeptide, constitutive androstane receptor (CAR) polypeptide, neuron-derived activated receptor (NOR1) polypeptide, nuclear receptor 1 (NURR1) polypeptide, estrogen receptor (ER) polypeptide, estrogen-related receptor (ERR) polypeptide, glucocorticoid receptor (GR) polypeptide, androgen receptor (AR) polypeptide, progesterone receptor (PR) polypeptide, or mineralocorticoid receptor (MR) polypeptide.

It will be appreciated that a nucleic acid or amino acid target sequence that aligns with a subject sequence can result in many different lengths with each length having its own percent identity. It is noted that the percent identity value can be rounded to the nearest tenth. For example, 78.11, 78.12, 78.13, and 78.14 is rounded down to 78.1, while 78.15, 78.16, 78.17, 78.18, and 78.19 is rounded up to 78.2. It is also noted that the length value will always be an integer.

The identification of conserved regions in a template, or subject, polypeptide can facilitate production of variants of wild type receptors. Conserved regions can be identified by locating a region within the primary amino acid sequence of a template polypeptide that is a repeated sequence, forms some secondary structure (e.g., helices and beta sheets), establishes positively or negatively charged domains, or represents a protein motif or domain. See, e.g., the Pfam web site describing consensus sequences for a variety of protein motifs and domains at http://www.sanger.ac.uk/Pfam/ and http://genome.wustl.edu/Pfam/. A description of the information included at the Pfam database is described in Sonnhammer et al, 1998, Nucl. Acids Res. 26: 320-322; Sonnhammer et al, 1997, Proteins 28:405-420; and Bateman et al, 1999, Nucl. Acids Res. 27:260-262. From the Pfam database, consensus sequences of protein motifs and domains can be aligned with the template polypeptide sequence to determine conserved region(s).

Conserved regions also can be determined by aligning sequences of the same or related polypeptides from closely related species. Closely related species preferably are from the same family. In some embodiments, alignment of sequences from two different species is adequate. For example, sequences from human and chimpanzee can be used to identify one or more conserved regions.

Typically, polypeptides that exhibit at least about 35% amino acid sequence identity are useful to identify conserved regions. Conserved regions of related proteins sometimes exhibit at least 40% amino acid sequence identity (e.g., at least 50%, at least 60%; or at least 70%, at least 80%, or at least 90% amino acid sequence identity). In some embodiments, a conserved region of target and template polypeptides exhibit at least 92, 94, 96, 98, or 99% amino acid sequence identity. Amino acid sequence identity can be deduced from amino acid or nucleotide sequence.

Also of interest are polypeptides that are mutants, fragments, and fusion of naturally occurring receptors provided that at least one ligand binding activity of a full-length naturally occurring receptor is retained. Typically, ligand binding activity of a mutant, fragment or fusion of a naturally occurring receptor will be at least 40% of the ligand binding activity of a wild type receptor, more typically between 50 to 80%; even more typically, between 70 to 90%; even more typically, more than 80% activity of the wildtype receptor.

A heterologous receptor polypeptide can have conservative substitutions, insertions, or deletions of a full-length naturally occurring coding sequence. One of skill will recognize that individual substitutions, deletions or additions to a polypeptide that alter, add or delete a single amino acid or a small percentage of amino acids in the encoded sequence is a “conservatively modified variant” where the alteration results in the substitution of an amino acid with a chemically similar amino acid. Conservative substitution tables providing functionally similar amino acids are well known in the art. The following six groups each contain amino acids that are conservative substitutions for one another:
1) Alanine (A), Serine (S), Threonine (T);
2) Aspartic acid (D), Glutamic acid (E);
3) Asparagine (N), Glutamine (Q);
4) Arginine (R), Lysine (K);
5) Isoleucine (I), Leucine (L), Methionine (M), Valine (V); and
6) Phenylalanine (F), Tyrosine (Y), Tryptophan (W).

In some instances, suitable receptors can be synthesized on the basis of consensus functional domains and/or conserved regions in polypeptides that are homologous receptors. Consensus domains and conserved regions can be identified by homologous polypeptide sequence analysis as described above. The suitability of such synthetic polypeptides for use as a heterologous receptor can be evaluated by functional complementation of a heterologous receptor polypeptide.

Alternatively, the heterologous receptor of the invention can be a fragment of a naturally occurring receptor. Usually, such fragment will comprise the ligand binding domain of the naturally occurring receptor. Domains are groups of substantially contiguous amino acids in a polypeptide that can be used to characterize protein families and/or parts of proteins. Such domains have a “fingerprint” or “signature” that can comprise conserved (1) primary sequence, (2) secondary structure, and/or (3) three-dimensional conformation.

Generally these domains have been correlated with specific in vitro and/or in vivo activities. A domain can have a length of from 10 amino acids to 100 amino acids, e.g., 10 to 50 amino acids, or 25 to 100 amino acids, or 35 to 65 amino acids, or 35 to 55 amino acids, or 45 to 60 amino acids. Typically, a nuclear hormone receptor polypeptide comprises a ligand binding domain, a DNA binding domain and a transactivation domain. Subregions in the amino acid sequence of a nuclear hormone receptor polypeptide can be designated, in order from N-terminus to C-terminus, as A/B, C, D, E, and F.

A. Ligand Binding Domain

A large C-terminal ligand binding domain (LBD) is typically observed in native nuclear hormone receptors, which generally have ligand contacts in three distinct clusters and separate from receptor dimerization contacts that also occur in the ligand binding domain. The conserved E1 subregion, as well as a less well-conserved heptad nine (h9) region and a second transactivation domain (AF2) also lie within the ligand binding domain.

A ligand binding domain generally has a tertiary structure which is a sandwich of 11 to 13 alpha-helices and several small beta-strands organized around a lipophilic binding cavity. Shown in Table 1 are specific amino acid sequences of ligand binding domains of naturally occurring receptors. Typically, a ligand binding domain can have conserved lysine, proline, phenylalanine, leucine, aspartic acid, and glutamine residues in the E1 subregion (Table 1 below). For example, the conserved amino acids in ER ligand binding domains are residues 362 (lysine), 365 (proline), 367 (phenylalanine), 370, 378, 379 (all leucine), 374 (aspartic acid), and 375 (glutamine). Typically, the ligand binding domain of the heterologous receptors of the invention can be mutants, fragments or fusions, which will exhibit conserved primary, secondary, and/or tertiary structural similarity to a member of the naturally occurring human nuclear hormone receptor family. In vitro or in vivo, a ligand binding domain of the heterologous receptor exhibits the ability to bind a known ligand of a nuclear hormone receptor. Typically, a heterologous receptor polypeptide retains the ability to bind a known ligand with a binding constant (Kd) of at least 300 nM, for example, a Kd of at least 200 nM, at least 100 nM, at least 75 nM, or at least 50 nM. Examples of ligand-binding domains are shown in Table 1 below.

TABLE 1

Conserved regions within ligand-binding domains of representative nuclear receptors.*

| | |
|---|---|
| Hs-M12674-ERα | HMINWAKRVPGFVDLTLHDQVHLLECAWLEILMIGLVWRSMEHPVKLL (SEQ ID NO:20) |
| Hs-478445-FXR | VLVEFTKKLPGFQTLDHEDQIALLKGSAVEAMFL--RSAEIFN--KKL (SEQ ID NO:21) |
| Hs-U22662-LXRα | EIVDFAKQLPGFLQLSREDQIALLKTSAIEVMLLETSRRYNPGSESIT (SEQ ID NO:22) |
| Hs-NM_005036-PPARα | ELTEFAKAIPGFANLDLNDQVTLLKYGVYEAIFAMLSSVMNKDGMLVA (SEQ ID NO:23) |
| Hs-NM_138712-PPARγ | EITEYAKSIPGFVNLDLNDQVTLLKYGVHEIIYTMLASLMNKDGVLIS (SEQ ID NO:24) |
| Hs-X06614-RARα | KTVEFAKQLPGFTTLTIADQITLLKAACLDILILRICTRYTPEQDTMT (SEQ ID NO:25) |
| Hs-NM_002957-RXRα | TLVEWAKRIPHFSELPLDDQVILLRAGWNELLIASFSHRSIAVKDGIL (SEQ ID NO:26) |
| Hs-BC002728-THRα | RVVDFAKKLPMFSELPCEDQIILLKGCCMEIMSLRAAVRYDPESDTLT (SEQ ID NO:27) |

*Identical amino acids are underlined.

B. DNA Binding Domain

A heterologous receptor polypeptide comprises a domain, termed a DNA binding domain, and also known as a “C domain,” that binds to a recognized site on DNA. A DNA binding domain typically contains two zinc-finger DNA binding motifs of the (Cys)4 type. In some embodiments, for example, thyroid receptor polypeptide, a variable C-terminal extension (CTE) flanks the zinc finger motifs and participates in DNA binding. Examples of DNA-binding domains are shown in Table 2 below.

TABLE 2

Conserved regions within DNA-binding domains of representative nuclear receptors.*

| | |
|---|---|
| Hs-M12674-ERα | TRYCAVCNDYASGYHYGVWSCEGCKAFFKRSIQGHN--DYMCPATNQCTIDKNRRKSCQACRLRKCYEVGMM |
| | (SEQ ID NO:28) |
| Hs-478445-FXR | DELCVVCGDRASGYHYNALTCEGCKGFFRRSITKNA--VYKCKNGGNCVMDMYMRRKCQECRLRKCKEMGML |
| | (SEQ ID NO:29) |
| Hs-U22662-LXRα | NELCSVCGDKASGFHYNVLSCEGCKGFFRRSVIKGA--HYICHSGGHCPMDTYMRRKCQECRLRKCRQAGMR |
| | (SEQ ID NO:30) |
| Hs-NM_005036-PPARα | NIECRICGDKASGYHYGVHACEGCKGFFRRTIRLKL--VYDK-CDRSCKIQKKNRNKCQYCRFNKCLSVGMS |
| | (SEQ ID NO:31) |
| Hs-NM_138712-PPARγ | AIECRVCGDKASGFHYGVHACEGCKGFFRRTIRLKL--IYDR-CDLNCRIHKKSRNKCQYCRFQKCLAVGMS |
| | (SEQ ID NO:32) |
| Hs-X06614-RARα | YKPCFVCQDKSSGYHYGVSACEGCKGFFRRSIQKNM--VUTCHRDKNCIINKVTRNRCQYCRLQKCFEVGMS |
| | (SEQ ID NO:33) |
| Hs-NM_002957-RXRα | KHICAICGDRSSGKHYGVYSCEGCKGFFKRTVRKDL--TYTCRDNKDCLIDKRQRNRCQYCRYQKCLAMGMK |
| | (SEQ ID NO:34) |
| Hs-BC002728-THRα | DEQCVVCGDKATGYHYRCITCEGCKGFFRRTIQKNLHPTYSCKYDSCCVIDKITRNQCQLCRFKKCIAVGMA |
| | (SEQ ID NO:35) |

*Identical amino acids are underlined.

A DNA binding domain of a heterologous receptor polypeptide will bind to a specific cis-responsive nucleotide sequence (Receptor Response Element) in a ligand dependent manner. The typical result is activation of transcription from a transcriptional start site associated with and operably linked to the receptor response element. Thus, activation of transcription from the transcription start site is ligand-dependent. In some embodiments, appropriate chaperonins or other components are necessary to facilitate binding of a DNA binding domain to its cognate receptor response element.

C. Transactivation Domain

A heterologous receptor polypeptide typically has discrete DNA binding and transactivation domains. Typically, transactivation domains, also known as A/B domains, can bring proteins of the cellular transcription and translation machinery into contact with the transcription start site to initiate transcription. A transactivation domain of a heterologous receptor polypeptide can be synthetic or can be derived from a source other than the ligand binding domain. Examples of suitable transactivation domains include the transactivation domains of herpes virus VP16 polypeptide and maize transcription factor C polypeptide.

D. Dimerization Sequences

In some embodiments, a heterologous receptor polypeptide comprises dimerization sequences. In some instances dimerization is required for the ligand/receptor complex to bind to its recognized DNA site. For example, PPAR is known to form a heterodimer with a retinoid X receptor (RXR) and binds to PPRE in the form of the heterodimer. Also, like other nuclear receptors, PPAR is considered to interact with a group of transcription coactivators to facilitate transcription activation activity.

Dimerization sequences can permit a receptor polypeptide to either produce homo- or heterodimers. Several motifs or domains in the amino acid sequence of a receptor can influence heterodimerization or homodimerization of a given nuclear receptor, e.g., the DNA binding (C) domain or the ligand binding (E) domain. The N-terminal part of the D domain (also known as the Hinge region) also can play a role in heterodimerization.

Thus, in some embodiments, a heterologous receptor described herein binds as a heterodimer to its cognate response element. In such embodiments, plant cells used in the method contain a coding sequence expressing a second receptor polypeptide, as a dimerization partner in addition to the coding sequence for the heterologous receptor polypeptide. A nuclear hormone receptor that binds as a heterodimer can be, for example, a retinoic acid receptor, thyroid receptor, vitamin D receptor, famesoid X receptor, oxysterol receptor, peroxisome proliferator receptor or ecdysone receptor, each of which bind as a heterodimer with the retinoid X receptor. With such receptors, plant cells used in the method contain a coding sequence expressing the retinoid X receptor, as a dimerization partner, in addition to the coding sequence for the heterologous receptor polypeptide. Other exemplary heterodimers include RXR/LXR, RXR/PXR, RXR/CAR, RXR/THR, RXR/THRα, RXR/THRβ, RXRα/THRβ, RXRα/THRα, RXRβ/THRα, RXRβ/THRβ and USP/EcR, RXRα/PPARγ, RXRα/PPARα, RXRα/RARα, RXRα/RARβ.

E. Localization Signals

A heterologous receptor polypeptide useful in the invention also can comprise a localization signal sequence, e.g., a cytoplasmic localization signal, a nuclear localization signal, an ER localization signal, a Golgi apparatus localization signal, or a cell membrane localization signal. It is recognized that a heterologous receptor may reside in the cytoplasm of a cell in the absence of ligand, translocating at least in part to the nucleus or other cellular compartment upon ligand-binding, as in the case of the glucocorticoid and mineralocorticoid receptors. A cytoplasmic localization signal can allow, for example, an increased ratio of cytoplasmic to nuclear localization.

Such a sequence can be an endogenous localization signal of a native nuclear hormone receptor or a synthetic sequence. An example of a cytoplasmic localization signal is a “membrane-anchoring domain,” which is a domain that can direct a heterologous receptor polypeptide to the cell cytoplasmic membrane. Suitable membrane-anchoring domains include, for example, a myristoylation (MYR) domain from, for example, a Src family kinase; a pleckstrin homology (PH) domain derived, for example, from an insulin receptor substrate, phopholipase C (PLC) or protein kinase B (PKB); or a C2 domain derived, for example, from protein kinase C (PKC) or a P13 kinase.

F. Epitope Tags

In some embodiments, a heterologous receptor polypeptide contains an epitope tag. Epitope tags can provide a convenient means for isolating a receptor polypeptide bound to a ligand and/or a receptor polypeptide unbound to a ligand or isolating the cell in the plant that is expressing the heterologous receptor polypeptide. A variety of epitope tags are known and used in the art including the FLAG® tag DYKDDDDK (SEQ ID NO:36), the HA tag YPYDVPDYA (SEQ ID NO:37), the c-Myc epitope EQKLISEEDL (SEQ ID NO:38), the AU1 tag DTYRYI (SEQ ID NO:39), and the 6-HIS tag HHHHHH (SEQ ID NO:40).

G. Chimeric Receptors

In other embodiments, a heterologous receptor polypeptide is a chimeric polypeptide. Examples include fusions of the ligand binding domain of one receptor and the DNA binding domain of another receptor. In another case, a chimeric construct can contain one or more domains of a full-length receptor polypeptide and one or more domains or motifs from a polypeptide that does not exhibit receptor activity, e.g., a ligand binding domain of a nuclear hormone receptor polypeptide, and DNA binding and transactivation domains of a viral non-hormone transcriptional activator polypeptide. In other embodiments, a heterologous receptor polypeptide is a chimeric polypeptide that contains a localization signal or an epitope tag. In certain instances, a VP16 activation domain can replace an activation domain of another receptor to result in a chimeric receptor polypeptide, for example, VP16THRβ.

Chimeric heterologous receptor polypeptides can also be useful for identifying ligands. Chimeric heterologous receptor polypeptides contain two or more polypeptide segments, each segment having one or more of the domains, tags, sequences or signals discussed above. Thus, a chimeric polypeptide can include a first polypeptide segment that exhibits a ligand binding activity of a nuclear hormone receptor, and a second polypeptide segment having the activities of DNA binding domain and a transactivator domain. In some embodiments, the first polypeptide segment exhibits 40% or greater (e.g., at least 40%, at least 60%, at least 80%, at least 90%, or at least 95%) sequence identity to the ligand binding domain of RXR, HFN4, testicular receptor, TLX, COUP-TF, THR, RAR, PPAR, reverb, ROR, SF-1, LRH-1, LXR, FXR, VDR, EcR, PXR, CAR, NOR1, NURR1, ER, ERR, GR, AR, PR, or MR. The first and second polypeptide segments are arranged such that a terminus of the second polypeptide segment is linked to a terminus of the first polypeptide segment via at least one covalent bond.

Typically, first and second polypeptide segments are directly linked via a peptide bond. In such embodiments the C-terminal amino acid of the first polypeptide segment can be linked to the N-terminal amino acid of the second polypeptide segment. Alternatively, the N-terminal amino acid of the first polypeptide segment can be linked to the C-terminal amino acid of the second polypeptide segment. In some embodiments, the first and second polypeptide segments can be indirectly linked via one or more (e.g., 1 to 50, and 10 to 50) intervening amino acids that are situated between the first and second polypeptides. In such embodiments, the C-terminal amino acid of the first polypeptide segment can be linked to an intervening amino acid, and the N-terminal amino acid of the second polypeptide segment can be linked to an intervening amino acid. Alternatively, the N-terminal amino acid of the first polypeptide segment can be linked to an intervening amino acid, and the C-terminal amino acid of the second polypeptide segment can be linked to an intervening amino acid. In some embodiments, the intervening amino acids include at least one alanine residue and/or at least one glycine residue.

In some embodiments, additional segments are present, e.g., a third segment having an epitope tag or a localization signal, or a first segment having a ligand binding domain as well as an epitope tag.

In some embodiments, a chimeric receptor polypeptide can include three segments. For example, a chimeric receptor polypeptide can include one segment having a ligand binding domain, a second segment having a DNA binding domain, and a third segment having a transactivation domain. In yet other embodiments a chimeric polypeptide can further include a fourth segment having dimerization sequences. In certain embodiments a chimeric polypeptide can further include a segment having a localization signal and a segment having an epitope tag. Segments in such chimeric receptors are linked as described above.

H. Heterologous Receptor Construct

Transgenic plant cells harbor a heterologous receptor construct which comprises a coding sequence and one or more regulatory sequences operably linked thereto. Coding sequences can be expressed to produce heterologous receptors comprising the domains described above. As used herein, nucleic acid refers to RNA or DNA, including cDNA, synthetic DNA or genomic DNA. The nucleic acids can be single- or double-stranded, and if single-stranded, can be either the coding or non-coding strand. As used herein with respect to nucleic acids, “isolated” refers to (i) a naturally-occurring nucleic acid encoding part or all of a polypeptide of the invention, but free of sequences, i.e., coding sequences, that normally flank one or both sides of the nucleic acid encoding polypeptide in a genome; (ii) a nucleic acid incorporated into a vector or into the genomic DNA of an organism such that the resulting molecule is not identical to any naturally-occurring vector or genomic DNA; or (iii) a cDNA, a genomic nucleic acid fragment, a fragment produced by polymerase chain reaction (PCR) or a restriction fragment. Specifically excluded from this definition are nucleic acids present in mixtures of nucleic acid molecules or cells.

Examples of suitable nucleic acids include nucleic acids encoding a human nuclear hormone receptor polypeptide. It will be appreciated that nucleic acids having a nucleotide sequence other than the specific nucleotide sequences disclosed herein still can encode a polypeptide having the exemplified amino acid sequence. The degeneracy of the genetic code is well known to the art; i.e., for many amino acids, there is more than one nucleotide triplet that serves as the codon for the amino acid. Furthermore, it is known that codons in a nucleic acid coding sequence can be selected for optimal expression in a particular species, if desired.

Recombinant nucleic acid constructs can contain cloning vector sequences in addition to other sequences described herein. Suitable cloning vector sequences are commercially available and are used routinely by those of ordinary skill. Nucleic acid constructs of the invention also can contain sequences encoding other polypeptides. Such polypeptides may, for example, facilitate the introduction or maintenance of the nucleic acid construct into a host organism. Other polypeptides also can affect the expression, activity, or biochemical or physiological effect of the encoded receptor polypeptide. Alternatively, other polypeptide coding sequences can be provided on separate nucleic acid constructs.

A nucleic acid encoding a heterologous receptor can be obtained by, for example, DNA synthesis or the polymerase chain reaction (PCR). PCR refers to a procedure or technique in which target nucleic acids are amplified. PCR can be used to amplify specific sequences from DNA as well as RNA, including sequences from total genomic DNA or total cellular RNA. Various PCR methods are described, for example, in PCR Primer: A Laboratory Manual, Dieffenbach, C. & Dveksler, G., Eds., Cold Spring Harbor Laboratory Press, 1995. Generally, sequence information from the ends of the region of interest or beyond is employed to design oligonucleotide primers that are identical or similar in sequence to opposite strands of the template to be amplified. Various PCR strategies are available by which site-specific nucleotide sequence modifications can be introduced into a template nucleic acid.

Nucleic acids can be detected by methods such as ethidium bromide staining of agarose gels, Southern or Northern blot hybridization, PCR or in situ hybridizations. Hybridization typically involves Southern or Northern blotting. Probes should hybridize under high stringency conditions to a nucleic acid or the complement thereof. High stringency conditions can include the use of low ionic strength and high temperature washes, for example 0.015 M NaCl/0.0015 M sodium citrate (0.1×SSC), 0.1% sodium dodecyl sulfate (SDS) at 65° C. In addition, denaturing agents, such as formamide, can be employed during high stringency hybridization, e.g., 50% formamide with 0.1% bovine serum albumin/0.1% Ficoll/0.1% polyvinylpyrrolidone/50 mM sodium phosphate buffer at pH 6.5 with 750 mM NaCl, 75 mM sodium citrate at 42° C.

I. Regulatory Elements

A coding sequence for a heterologous receptor is operably linked to one or more regulatory elements that facilitate transcription and translation of the receptor coding sequence. Typically, the receptor coding sequence is constitutively expressed, e.g., via a CaMV 35S promoter. However, expression may be made inducible for those instances in which constitutive expression results in undesirable physiological or morphological effects such as death or slow growth.

Regulatory elements can include promoter sequences, enhancer sequences, insulator elements, response elements, protein recognition sites, inducible elements that modulate expression of a nucleic acid sequence, promoter control elements, protein binding sequences, 5′ and 3′ UTRs, transcriptional start sites, termination sequences, polyadenylation sequences, introns and certain sequences within amino acid coding sequences such as secretory signals, and protease cleavage sites. As used herein, “operably linked” refers to positioning of a regulatory element in a construct relative to a nucleic acid in such a way as to permit or facilitate transcription and/or translation of the nucleic acid. The choice of element(s) to be included depends upon several factors, including, but not limited to, replication efficiency, selectability, inducibility, desired expression level, and cell or tissue specificity.

Typically, a promoter is located 5′ to the sequence to be transcribed, and proximal to the transcriptional start site of the sequence. Promoters are upstream of the first exon of a coding sequence and upstream of the first of multiple transcription start sites. In some embodiments, a promoter is positioned about 3,000 nucleotides upstream of the ATG of the first exon of a coding sequence. In other embodiments, a promoter is positioned about 2,000 nucleotides upstream of the first of multiple transcription start sites. The promoters of the invention comprise at least a core promoter. Additionally, the promoter may also include at least one control element such as an upstream element. Such elements include upstream activation regions (UARs) and optionally, other DNA sequences that affect transcription of a polynucleotide such as a synthetic upstream element.

A 5′ untranslated region (UTR) is transcribed, but is not translated, and lies between the start site of the transcript and the translation initiation codon and includes the +1 nucleotide. A 3′ UTR can be positioned between the translation termination codon and the end of the transcript. UTRs can have particular functions such as increasing mRNA message stability or translation attenuation. Examples of 3′ UTRs include, but are not limited to polyadenylation signals and transcription termination sequences.

In some embodiments, regulatory elements that preferentially drive transcription in specific cell types, tissues, or developmental stages are used. For example, a promoter that drives transcription in cells from vegetative tissue can be used, e.g., leaf cells or root cells. A cell type or tissue-specific promoter is sometimes observed to drive expression of operably linked sequences in tissues other than the target tissue. Thus, as used herein a cell type or tissue-specific promoter is one that drives expression preferentially in the target tissue, but can also lead to some expression in other cell types or tissues as well. Methods for identifying and characterizing regulatory elements in plant genomic DNA are known.

II. Reporter Construct

Plant cells for use in the methods described herein typically contain a reporter construct. A reporter construct comprises a cis-responsive receptor element (receptor response element, RRE) for the heterologous receptor/ligand complex to bind, and a sequence to be transcribed, which is operably linked to the RRE. Interaction between a candidate ligand and a heterologous receptor polypeptide results in activation of transcription from a promoter operably linked to the RRE and expression of the sequence to be transcribed. A sequence to be transcribed can be a coding sequence of a protein that exhibits a readily detectable property. Alternatively, a sequence to be transcribed can be an antisense, RNAi or sense suppression construct that inhibit a reporter gene. Thus, reporter activity is responsive to modulation by the ligand of signal transduction activity of the heterologous receptor. It will be appreciated that stability of a particular reporter may vary among species, or among cultivars of the same species. In addition, other factors may affect reporter coding sequence stability and expression levels including, but not limited to, transformation conditions and methods. Thus, a suitable reporter coding sequence can be selected for a given use.

A. Receptor Response Elements (RRE)

Cis-responsive receptor elements are known for many nuclear hormone receptor polypeptides, and typically include a hexanucleotide sequence as half of the element. The half-element often is a variation of the hexanucleotide AGGTCA, although several receptor polypeptides such as glucocorticoid receptor, mineralocorticoid receptor, progesterone receptor and androgen receptor bind an AGAACA half-site. Exemplary sequences for cis-acting receptor response elements are shown in Table 3 below.

TABLE 3

| | | | |
|---|---|---|---|
| Receptor (Response Element) | Element Type | Sequence | SEQ ID NO: |

| | | | |
|---|---|---|---|
| GR/MR/PR/AR | Inverted repeat | AGAACAnnnTGTTCT | 41 |
| ER (ERE) | Inverted repeat | AGGTCAnnnTGACCT | 42 |
| RXR/PPAR, RXR/RXR, RAR/RXR, | Direct Repeat | AGGTCAnAGGTCA | 43 |
| RXRα/THRβ (RXRE) | | | |
| RXR/RAR (RARE) | Direct Repeat | AGGTCAnnAGGTCA | 44 |
| RXR/VDR (VDRE) | Direct Repeat | AGGTCAnnnAGGTCA | 45 |
| RXR/THR, THRβ (TRE) | Direct Repeat | AGGTCAnnnnAGGTCA | 46 |
| RXR/RAR (RARE) | Direct Repeat | AGGTCAnnnnnAGGTCA | 47 |

When a heterologous receptor polypeptide with ligand binds as a heterodimer to its cognate receptor response element, binding can occur in one of several patterns. Some receptors bind as a heterodimer on directly (tandemly) repeated half response elements. The tandem repeats typically are separated by about 1-5 nucleotides. Alternatively, binding as a heterodimer occurs on inverted (palindromic) response elements separated by 1 base pair. RXR/RAR, RXR/VDR, RXR/LXR, RXR/PXR, RXR/CAR, PPAR/RXR, and RXR/PPAR heterodimers bind to direct repeats, whereas RXR/THR and USP/EcR bind to inverted half-repeats. One or more of such repeats constitute a receptor response element. Typically, a cis responsive receptor element contains 1-5 repeats, but may contain more than 5 repeats (e.g., 7, 8, 9, 10, 12, 15, 17, 18, 19 or 20).

In other embodiments, a heterologous receptor used in a method described herein binds as a homodimer to its cognate response element. Such a heterologous receptor can be, for example, a glucocorticoid, estrogen, androgen, progestin, or mineralocorticoid receptor. When a heterologous receptor polypeptide with ligand binds as a homodimer to its cognate receptor response element, binding occurs as a homodimer on inverted repeats separated by 3 base pairs, as a homodimer on direct repeats separated by 1 bp, or as a monomer on a single half-site, which may contain a 5′ extension of 2 nucleotides. While both receptors of a homodimer likely are liganded for activity, ligand binding of the primary receptor residing on the 3′ half-element generally is sufficient for activity of a heterodimer.

B. Sequence to be Transcribed

Typically, a sequence to be transcribed is a coding sequence for a reporter polypeptide. There are a number of suitable reporter polypeptides whose coding sequence can be operably linked to one or more receptor response elements. For example, a polypeptide that, when expressed by a cell, emits fluorescence upon exposure to light of an appropriate excitation wavelength is suitable. Such polypeptides include green fluorescent protein (GFP), red fluorescent protein (RFP) and yellow fluorescent protein (YFP). Such fluorescent proteins can be a wild-type GFP derived from the jelly fish Aequorea victoria, or from other members of the Coelenterata, such as the red fluorescent protein from Discosoma spp., GFP from Renilla reniformis, GFP from Renilla muelleri or fluorescent proteins from other animals, fungi or plants. Polypeptides modified from the amino acid sequence found in nature can also be used, e.g., modifications such as a blue fluorescent variant of GFP; modifications that change the spectral properties of GFP fluorescence, or modifications that exhibit increased fluorescence when expressed in cells at a temperature above about 30° C. See, e.g., WO 97/11094. Examples of GFP variants are F64L-GFP, F64L-Y66H-GFP F64L-S65T-GFP, and F64L-E222G-GFP. Some variants are commercially available from Invitrogen or from Clontech. See also, GenBank Acc. Nos. U55762 and U55763. Other suitable reporter polypeptides include β-galactosidase, β-glucuronidase (GUS), firefly luciferase, and chimeric polypeptides comprising an epitope tag, a membrane localizing segment and a reporter segment. See, e.g., U.S. patent application Ser. No. 10/046,660. A reporter polypeptide can also be a polypeptide that modulates calcium flux or induces expression of an endogenous receptor.

In some embodiments, a sequence to be transcribed encodes a chimeric reporter polypeptide. For example, a chimeric reporter polypeptide may contain a reporter segment, and a segment having an epitope tag. A variety of epitope tags are known and used in the art including the FLAG® tag (DYKDDDDK; SEQ ID NO:36), the HA tag (YPYDVPDYA; SEQ ID NO:37), the c-Myc epitope (EQKLISEEDL; SEQ ID NO:38), the AU1 tag (DTYRYI; SEQ ID NO:39), and the 6-HIS tag (HHHHHH; SEQ ID NO:40). In certain cases, a segment containing an epitope tag is located adjacent to the N-terminus of a reporter segment. In other cases, a segment containing an epitope tag is located adjacent to the C-terminus of a reporter segment. For example, a chimeric luciferase reporter polypeptide can include an HA tag at its N-terminus, and a chimeric GFP reporter polypeptide can include a 6-HIS tag at its C-terminus. In other cases, a chimeric polypeptide contains a reporter segment, and two additional segments, each additional segment containing an epitope tag.

III. Transgenic Plants and Plant Cells

Plants and plant cells useful in the methods described herein contain a nucleic acid encoding a heterologous receptor polypeptide and, optionally, a nucleic acid encoding a reporter polypeptide. Such nucleic acids can be introduced into plant cells on separate constructs or on the same construct. Generally, a nucleic acid molecule is in the form of a plasmid. The compositions of, and methods of constructing, nucleic acid molecules for successful transformation of plants are known to those skilled in the art, e.g., the use of suitable nucleic acid components such as promoters, polyadenylation sequences, selectable marker sequences, enhancers, introns, and the like. Cells that integrate an introduced nucleic acid sequence into their genome are called stably transformed cells. Stably transformed cells typically retain the introduced nucleic acid sequence with each cell division. Cells containing introduced nucleic acids that are not integrated into the genome are called transiently transformed cells. Transiently transformed cells typically lose some portion of the introduced nucleic acid sequence with each cell division such that the introduced nuclei acid cannot be detected in daughter cells after sufficient number of cell divisions. Thus, transformed cells can be either transiently and/or stably transformed. Both transiently transformed and stably transformed transgenic plant cells can be useful in the methods described herein.

Transgenic plant cells growing in suspension culture, or tissue or organ culture, can be used to identify ligands that interact with receptor polypeptides. For the purposes of this invention, solid and/or liquid tissue culture techniques can be used. When using solid medium, transgenic plant cells can be placed directly onto the medium or can be placed onto a filter film that is then placed in contact with the medium. When using liquid medium, transgenic plant cells can be placed onto a floatation device, e.g., a porous membrane, that contacts the liquid medium. Solid medium typically is made from liquid medium by adding agar. For example, a solid medium can be Murashige and Skoog (MS) medium containing agar and a suitable concentration of an auxin, e.g., 2,4-dichlorophenoxyacetic acid (2,4-D), and a suitable concentration of a cytokinin, e.g., kinetin. In some embodiments, transgenic plant cells are protoplasts.

In other embodiments, transgenic plant cells suitable for identifying ligands that interact with receptor polypeptides constitute part or all of a whole plant. Transgenic plants can be bred prior to use in methods disclosed herein, e.g., to introduce a nucleic acid into other lines, to transfer a nucleic acid to other species or for further selection of other desirable traits. Alternatively, transgenic plants can be propagated vegetatively for those species amenable to such techniques. Progeny includes descendants of a particular plant or plant line. Progeny of an instant plant include seeds formed on F₁, F₂, F₃, and subsequent generation plants, or seeds formed on BC₁, BC₂, BC₃, and subsequent generation plants.

Seeds produced by a transgenic plant can be grown and then selfed (or outcrossed and selfed) to obtain seeds homozygous for the nucleic acid encoding a heterologous receptor polypeptide.

When transiently transformed plant cells are used, an assay for reporter activity can be performed at a suitable time after transformation. A suitable time for conducting the assay typically is about 1-21 days after transformation, e.g., about 1-14 days, about 1-7 days, or about 1-3 days. The use of transient assays is particularly convenient for rapid analysis of candidate ligands in different species, or to confirm expression of a heterologous receptor whose expression has not previously been confirmed in the recipient cells. In some cases, leaf tissue is suitable for conducting a transient assay, for example, leaf disks. In other cases, other plant tissue such as roots, root hairs, pollen, or seed tissue, is suitable.

Techniques for introducing exogenous nucleic acids into monocotyledonous and dicotyledonous plants are known in the art, and include, without limitation, Agrobacterium-mediated transformation, viral vector-mediated transformation, electroporation and particle gun transformation, e.g., U.S. Pat. Nos. 5,538,880, 5,204,253, 6,329,571 and 6,013,863. If a cell or tissue culture is used as the recipient tissue for transformation, plants can be regenerated from transformed cultures if desired, by techniques known to those skilled in the art.

A suitable group of plant species include dicots, such as safflower, alfalfa, soybean, rapeseed (high erucic acid and canola), or sunflower. Also suitable are monocots such as corn, wheat, rye, barley, oat, rice, millet, amaranth or sorghum. Other suitable species include Catharanthus roseus, Vinca major, Eschscholtzia californica, Papaver spp. (e.g., P. somniferum,) Camptotheca accuminata, Rauwolfia spp., Digitalis spp., Mentha spicata, M pulegium, M piperita, Thymus vulgaris L., Orikanum vulgare, Rosmarinus officinalis, Melissa officinalis, Lavandula augustifolia or Salvia officinalis. Also suitable are vegetable crops or root crops such as broccoli, peas, sweet corn, popcorn, tomato, beans (including kidney beans, lima beans, dry beans, green beans) and the like. Also suitable are fruit crops such as peach, pear, apple, cherry, orange, lemon, grapefruit, plum, mango and palm. Thus, the invention has use over a broad range of plants, including species from the genera Arabidopsis, Agastache Anacardium, Arachis, Asparagus, Atropa, Avena, Brassica, Citrus, Citrullus, Capsicum, Catharanthus, Carthamus, Cocos, Coffea, Cucumis, Cucurbita, Datura, Daucus, Elaeis, Echinacea, Eschscholtzia, Fragaria, Glycine, Gossypium, Helianthus, Heterocallis, Hordeum, Hyoscyamus, Hyssopus, Lactuca, Linum, Lolium, Lupinus, Lycopersicon, Malus, Manihot, Majorana, Medicago, Mentha, Nicotiana, Ocimum, Olea, Oenothera, Oryza, Panicum, Pannesetum, Papaver, Persea, Phaseolus, Pinus, Pistachia, Pisum, Plantago, Pyrus, Prunella, Prunus, Raphanus, Rheum, Ricinus, Salvia, Secale, Senecio, Sinapis, Solanum, Sorghum, Theobromus, Trifolium, Trigonella, Triticum, Vicia, Vinca, Vitex, Vitis, Vigna, Withania, and Zea.

A. Heterologous Signal Transduction Polypeptides

In some embodiments, transgenic plants also can include a heterologous signal transduction polypeptide that can interact with the heterologous receptor to mediate the heterologous receptor's signal transduction activity. Such polypeptides include transcription co activators and chaperonins. In some embodiments, a heterologous signal transduction polypeptide is a chimera of a non-plant signal transduction polypeptide and a plant signal transduction polypeptide.

IV. Candidate Ligands/Reporter Activity

A. Detection of Reporter Activity

A candidate ligand is identified when an increase in reporter activity is detected in transgenic plant cells described herein. Reporter activity is determined after one or more ligands contact the receptor polypeptide. If there is an interaction between a ligand and the receptor polypeptide, signal transduction activity of the receptor is modulated, resulting in a change in reporter activity. Interaction typically occurs by specific binding between the ligand and the receptor polypeptide. Typically, a heterologous receptor polypeptide/ligand complex results in activation of transcription from a promoter operably linked to the cognate RRE and in translation of a reporter polypeptide encoded by the resulting transcript.

Detection of reporter activity will vary with the reporter used. For example, when GFP is used as a reporter, emitted light can be measured with various apparatus known to the person skilled in the art. Typically, such apparatus comprises a light source, a method for selecting the wavelength(s) of light from the source that will excite the luminescence of the luminophore, a device that can rapidly block or pass the excitation light into the rest of the system, a series of optical elements for conveying the excitation light to the specimen, collecting the emitted fluorescence in a spatially resolved fashion, and forming an image from this fluorescence emission (or another type of intensity map relevant to the method of detection and measurement), a detector to record the light intensity, preferably in the form of an image, and a computer or electronic system and associated software to acquire and store the recorded information and/or images, and to compute the degree of redistribution from the recorded images. In one embodiment, reporter activity is detected using a fluorescence microscope. In another embodiment, reporter activity is detected using a charged coupled device (CCD) camera. An apparatus system can be automated. Alternatively, reporter activity can be determined in a qualitative manner, e.g., by visual observation of fluorescence intensity under a microscope. In some embodiments, reporter activity after contacting one or more candidate ligands and a receptor polypeptide is compared to reporter activity in a control, e.g., reporter activity in the absence of any contact between a ligand and a receptor polypeptide.

Candidate ligands can be compounds synthesized by the transgenic plant cells expressing a heterologous receptor polypeptide. Such ligands can be considered endogenous ligands. A candidate ligand may be a small organic compound or a biopolymer such as a protein or peptide.

In some embodiments, endogenous ligands are compounds synthesized by the same plant cells as those expressing a heterologous receptor. In other embodiments, endogenous ligands are compounds synthesized by plant cells other than the transgenic cells expressing the heterologous receptor. For example, endogenous ligands can be synthesized in a first tissue of a plant, e.g., a root tissue or leaf tissue, whereas a heterologous receptor is expressed preferentially in a second tissue of the plant, e.g., a meristematic tissue or floral tissue. Endogenous ligands are transported in the plant to the second tissue, are taken up by cells of the second tissue, and may lead to an increase in reporter activity. As another example, a first transgenic plant tissue or organ culture can be grown, such as a feeder layer, in the presence of a second plant tissue or organ culture. Endogenous ligands from the first tissue or organ culture can diffuse in media, be taken up by cells of the second tissue or organ culture and may lead to an increase in reporter activity. It will be appreciated that the first tissue or organ may or may not express the heterologous receptor. Plant cells in which reporter activity is detected can be subjected to fractionation to characterize ligand(s) responsible for reporter activity. Alternatively, plant cells known or suspected of synthesizing endogenous ligands can be subjected to fractionation to characterize ligand(s) responsible for reporter activity.

In some embodiments, plant cells are subjected to environmental conditions that facilitate the synthesis of increased amounts of endogenous ligands. Environmental conditions under which a plant, or a plant or cell culture, is grown can be altered, e.g., by increasing the temperature, increasing the watering rate, or decreasing the watering rate, relative to a control temperature or watering rate. Other environmental conditions that can be altered in order to increase the amount or synthesis rate of endogenous ligands include the concentration of salt, minerals, hormones, nitrogen, carbon, osmoticum, or known elicitors such as yeast extract, salicylic acid, and methyl jasmonate.

In other embodiments, one or more candidate ligands are present in an extract. Suitable sources include extracts from a plant cell type, tissue or organ. Such extracts can be crude extracts, or can be partially purified, or extensively purified. Such extracts can be aqueous extracts or non-aqueous extracts. Suitable tissues or organs from which to prepare plant extracts include leaves, roots, stems, bark, flowers, seeds, embryos, endosperm, cotyledons, trichomes, meristematic tissue, embryogenic cultures, organogenic cultures, or cambial cells. Such extracts can be permitted to contact plant cells expressing the heterologous receptor polypeptide.

B. Ligand Synthesis Polypeptides

In some embodiments, candidate ligands are obtained from plant cells comprising a recombinant nucleic acid construct encoding a polypeptide that mediates the synthesis of increased amounts of naturally-occurring endogenous compounds, or mediates the synthesis of novel endogenous compounds. Such compounds are candidate ligands that can be screened for their activity as described herein.

For example, genes encoding polypeptides involved in sterol biosynthesis can be overexpressed in a plant, resulting in increased amounts of sterols, and/or synthesis of novel sterols. Such increased or novel sterols can be screened for their activity as ligands for steroid receptors. Polypeptides involved in sterol biosynthesis include squalene synthase polypeptides, e.g., the polypeptides encoded by the SQS1 and SQS2 genes from Arabidopsis, tomato, and Brassica. Other polypeptides suitable for increasing sterol amounts and/or producing novel sterols include, without limitation, lupeol synthase genes, cycloartenol synthase genes, sterol methyl oxidase genes, and regulatory factor genes controlling the expression of the above genes and genes involved in the sterol biosynthetic pathway.

As another example, genes encoding polypeptides involved in flavonoid biosynthesis can be overexpressed in a plant, resulting in increased amounts of flavonoids, and/or synthesis of novel flavonoids. Such increased or novel flavonoids can be screened for their activity as ligands for flavonoid receptors. Isoflavones are potential phytoestrogens and are potential ligands for estrogen receptors. Polypeptides involved in flavonoid biosynthesis include but are not limited to chalcone isomerase genes, chalcone reductase genes, dihydroflavonol reductase genes, isoflavone synthase genes, isoflavone reductase genes, flavonoid 3-hydroxylase genes, and regulatory factor genes controlling the expression of the above genes and genes involved in flavonoid biosynthetic pathways.

In some instances, genes encoding polypeptides involved in biosynthesis of phenolics can be overexpressed in a plant, resulting in increased amounts of phenolic compounds, including, for example, anthocyanins, coumarins, and psoralens. Overexpression of such genes also can lead to synthesis of novel phenolics. Such increased or novel phenolics can be screened for their activity as ligands for phenolic receptors.

In some cases, genes encoding polypeptides involved in terpenoid biosynthesis can be overexpressed in a plant, resulting in increased amounts of terpenoids, and/or synthesis of novel terpenoids. Such increased or novel terpenoids can be screened for their activity as ligands for terpenoid receptors. In some embodiments, genes encoding polypeptides involved in alkaloid biosynthesis can be overexpressed in a plant, resulting in increased amounts of alkaloids, and/or synthesis of novel alkaloids. Such increased or novel alkaloids can be screened for their activity as ligands for alkaloid receptors.

In yet other cases, genes encoding polypeptides involved in biosynthesis of polythienyls, isothiocyanates, glucosinolates, cyanogenic glycosides, polyacetylenes, lipids, sesquiterpenoids, or quassinoids can be overexpressed in a plant, resulting in increased amounts of such compounds, and/or synthesis of novel compounds.

Nucleic acids encoding such polypeptides can be under the control of a constitutive promoter or inducible promoter, e.g., a promoter that confers increased levels of transcription in response to an increase in temperature or the presence of an inducer. Plant cells comprising such genes can be the same as, or different from, the plant cells that comprise a nucleic acid encoding a heterologous receptor.

C. Fractionation

Plant cells in which reporter activity is detected, or plant cells known or suspected of synthesizing endogenous ligands, can be subjected to fractionation to characterize the ligand(s) responsible for reporter activity. Typically, fractionation is guided by in vivo reporter activity of fractions or by in vitro assay of fractions. In some instances, cells exhibiting reporter activity, and which contain one or more candidate ligands, can be separated from cells not exhibiting reporter activity. Such a separation can enrich for cells or cell types that contain such ligands. A number of methods for separating particular cell types or cell layers are known to those having ordinary skill in the art. For example, cell types exhibiting reporter activity may be dissected using laser capture microdissection, or can be captured using a cell sorter by virtue of an epitope tag in the reporter or receptor.

Fractionation can be carried by techniques known in the art. For example, samples can be extracted with 100% MeOH to give a crude oil which is partitioned between several solvents in a conventional manner. As an alternative, hexane and methylene chloride fractionation can be carried out on gel columns using methylene chloride and ethyl acetate/hexane solvents.

In vitro assays for identifying candidate ligands can be based on the transcription activity of the heterologous receptor of interest. In other embodiments, an in vitro assay is based on an assay that measures the ability of compounds in a fraction to specifically bind to the heterologous receptor of interest, e.g., in a competitive homogeneous biochemical assay. For example, a plant tissue or organ suspected to contain ligands can be fractionated, and the effluent of the fractionation contacted with a predetermined amount of receptor suspected to bind such ligands. A predetermined amount of a known ligand(s) that binds to the receptor is added under conditions in which complexes between the known ligand and the receptor can form in the mixture. The amount of known ligand in the mixture that is unbound is detected using, for example, a fluorescent label on the known ligand or a known ligand that has intrinsic fluorescence.

In other embodiments, a fractionated or unfractionated plant tissue or organ is subjected to mass spectrometry in order to identify and characterize candidate ligand(s). See, e.g., WO 02/37111. Mass spectrometry analysis is often suitable for characterizing and identifying particular ligands that are responsible for reporter activity. In some embodiments, electrospray ionization (ESI) mass spectrometry can be used. In other embodiments, atomospheric pressure chemical ionization (APCI) mass spectrometry is used. If it is desired to identify higher molecular weight molecules in an extract, matrix-assisted laser desorption/ionization time-of-flight (MALDI-TOF) mass spectrometry can be useful.

Methods described herein can facilitate more rapid identification of candidate ligands compared to other types of ligand-identification methods. Methods disclosed herein permit screening of a wide-ranging group of plants known or suspected of containing ligands and, if desired, permit screening to be focused on particular tissues or organs in such plants. In contrast, many known methods are more difficult to practice with particular tissues or organs and lack the sensitivity attainable with the novel methods described herein.

Novel ligands to nuclear receptors can be used, for example, as pharmaceuticals or diagnostics to treat or detect cancer. Breast cancer and prostate cancer cells have been shown to express estrogen and testosterone receptors, respectively. Novel ligands to such receptors can be conjugated to radioisotopes to deliver targeted radiotherapy to breast and prostate cancer cells. Also, novel ligands to such receptors can be conjugated to fluorophores to detect increased levels of estrogen and testosterone receptors, which may be related to an increase in the number of cancerous breast or prostate cells in a patient. Novel ligands can also be used as the basis for synthesizing analogs of the novel ligand. Such analogs may possess new or modified therapeutic activity.

The invention will be further described in the following examples, which do not limit the scope of the invention described in the claims.

### EXAMPLES

### Example 1

### VP16ER Nucleic Acid Constructs and Transformation

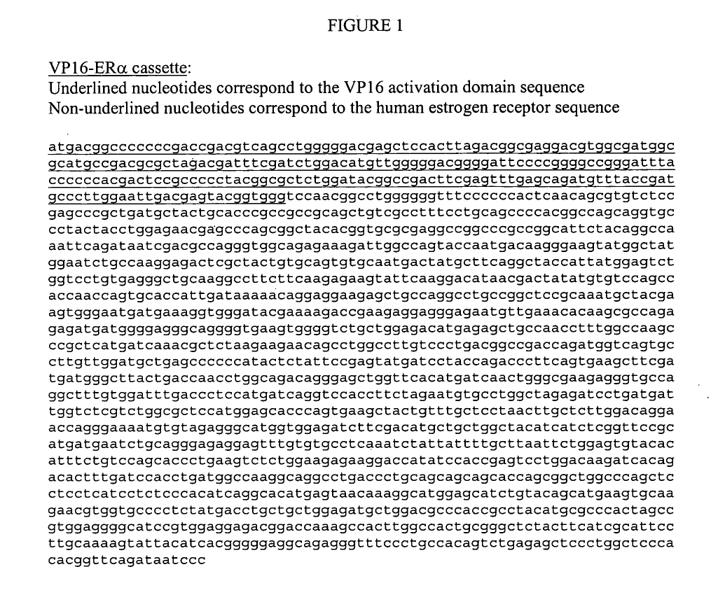

A receptor nucleic acid construct was made comprising the coding sequence for a VP16-human estrogen receptor alpha (ERa) chimeric polypeptide operably linked to a CaMV35S promoter and a nopaline synthase (NOS) terminator. The VP16 activation domain replaces the first 90 amino acids of the human ERα polypeptide. The coding sequence for the chimeric polypeptide is shown in FIG. 1 and SEQ ID NO: 1. The amino acid sequence is shown in SEQ ID NO: 2. Underlined nucleotides correspond to the VP16 activation domain sequence, and non-underlined nucleotides correspond to the human estrogen receptor sequence.

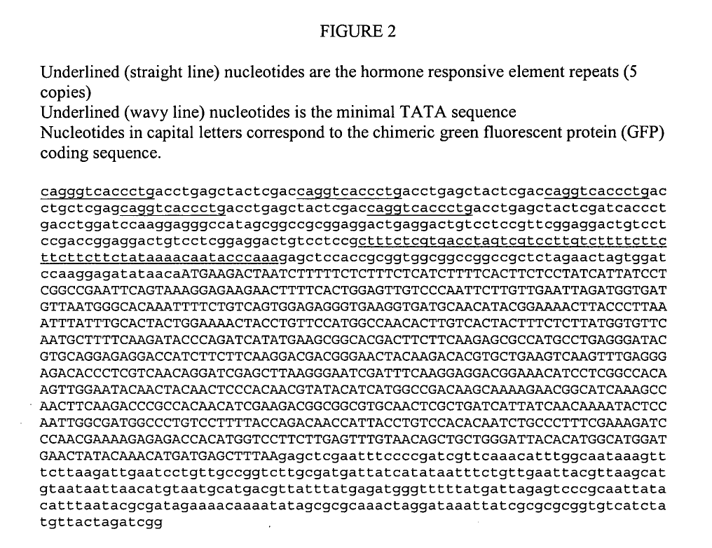

A reporter nucleic acid construct was made comprising the coding sequence for a chimeric green fluorescent protein operably linked to five copies of a human estrogen response element. The nucleotide sequence for the ERE-GFP construct is shown in FIG. 2 and SEQ ID NO: 3. The five copies of the estrogen response element are underlined. The minimal TATA sequence is indicated by a wavy underline. Nucleotides in capital letters are the coding sequence for the chimeric green fluorescent protein (GFP), which contains a chitinase signal sequence at the 5′-end and an HDEL (SEQ ID NO:48) signal sequence at the 3′-end. The amino acid sequence is shown in SEQ ID NO: 4.

The VP16ERα construct and the ERE-GFP construct were cloned into an Agrobacterium binary vector to make a vector designated Bin4-EUTG-10::VPCR-ER-1#81. The vector contains the 28716 promoter driving expression of a synthetic phosphinothricin resistance gene, followed by an octopine synthase (OCS) terminator. The phosphinothricin resistance gene permits selection of transformed plant cells. The vector also contains a spectinomycin resistance gene for selection in Agrobacterium. A control Agrobacterium binary vector, designated Bin4-EUTG-101, was also made. Bin4-EUTG-101 contains the phosphinothricin resistance and spectinomycin resistance genes, but lacks the VP16ERα and ERE-GFP sequences. Transgenic plants carrying Bin4-EUTG-101 were used as controls.

Transformation of rice was carried out in a manner similar to that described in U.S. Pat. No. 5,591,616. Transformants were selected using a phosphinothricin resistance gene and bialaphos as the selective agent. Transformation of Arabidopsis was carried out essentially as described in Bechtold et al., C.R. Acad. Sci. Paris, 316:1194-1199 (1993). Seeds formed on primary transformants are referred to as the T1 generation, and plants germinated from such seeds are referred to as T1 plants.

### Example 2

### Transgenic Rice Containing a VP16ER Construct

Transgenic rice callus containing the Bin4-EUTG-10::VPCR-ER-1#81 vector was made. About 50 selected lines were obtained and cultured at 27° C. on N6 media containing 5 mg/ml bialaphos, with transfer to fresh media occurring about every 2 weeks. Callus from 6 lines, at about 45 days after transformation, was incubated with about 5 uM estradiol or 5 uM 4-hydroxytamoxifen. After incubation for about 40 hours, callus cells were examined under a dissecting microscope at 10× to 60× magnification.

Four out of 6 lines examined, lines 1, 2, 5 and 6, showed moderate to very bright green fluorescence in the presence of the exogenous compounds, but no fluorescence in the absence of the exogenous compounds. The results of these experiments indicate that exogenously applied estradiol or 4-hydroxytamoxifen can activate transcription of the GFP coding sequence present in these four transgenic lines.

Two of the 6 rice callus lines examined showed weak to moderate green fluorescence even in the absence of the exogenous compounds, and moderate to bright green fluorescence in the presence of the exogenous compounds. The results indicate that these two lines have endogenous compounds that activate transcription of the GFP coding sequence present in Bin4-EUTG-10::VPCR-ER-1#81 under these culture conditions.

### Example 3

### Transgenic Arabidopsis Containing a VP16ER Construct

Transgenic Arabidopsis plants containing the Bin4-EUTG-10::VPCR-ER-1#81 vector were made as describe in Example 1. About 100 transgenic T2 seeds from each of 3 independent T1 plants were germinated at 22° C. on MS agar medium alone, or on MS agar medium containing about 5 uM estradiol or 5 uM 4-hydroxytamoxifen. PCR analysis indicated that receptor construct sequences were present in the seedlings in heterozygous and homozygous conditions. The seedlings were examined under a dissecting microscope for green fluorescence starting at 5 days after germination. All of the T2 seedlings grown in the presence of estradiol or 4-hydroxytamoxifen exhibited moderate to strong green fluorescence, whereas none of the T2 seedlings grown in the absence of estradiol or 4-hydroxytamoxifen exhibited green fluorescence.

An isoflavone extract was prepared from dried soybean seeds by grinding the seeds in a mill grinder under liquid nitrogen. About 3 grams of ground seeds was extracted with methanol for about 10 min at room temperature. The methanol extract was then incubated with ethyl acetate for about 10 min at room temperature. Ethyl acetate soluble compounds were concentrated by vacuum drying. The dried pellet was resuspended in 200 μl DMSO and the crude isoflavone extract was stored at 4° C. The extract was used within 2 days of preparation.

About 100 T2 seeds from the 3 T1 plants described above were germinated at 22° C. on MS agar medium containing about 100 μl isoflavone extract/350 ml media. T2 seeds were also germinated on MS agar medium containing 100 μl DMSO/350 ml media as a control. Seedling roots were examined under a dissecting microscope for green fluorescence starting at 5 days after germination. All of the T2 seedlings grown in the presence of extract exhibited moderate to strong green fluorescence. None of the T2 seedlings grown in the absence of the extract exhibited green fluorescence. T2 transgenic Arabidopsis plants containing the Bin4-EUTG-101 control construct exhibited no green fluorescence either in the presence or absence of the extract. These results indicate that the soybean isoflavone extract contains compounds that activate transcription of the GFP coding sequence present in the Bin4-EUTG-10::VPCR-ER-1#81 transgenic plants.

### Example 4

### Transgenic California Poppy Containing a VP16ER Construct

Root explants from aseptically germinated California poppy seedlings were cocultivated with Agrobacterium containing the Bin4-EUTG-10::VPCR-ER-1#81 vector. After 2 days, treated root explants were rinsed with liquid tissue culture medium to remove Agrobacterium and transferred to callus inducing medium (CIM) containing 0.5 mg/ml auxin, 0.5 mg/ml cytokinin, and 2.5 mg/ml bialaphos. More than 20 callus clusters that appeared to be surviving in the presence of bialaphos (putative transformants) were examined under a dissecting microscope at 10× to 60× magnification starting at 3 days after transfer to CIM. Several of the callus clusters examined exhibited moderate to strong green fluorescence. None of the explants from control plants transformed with the Bin4-EUTG-101 control construct exhibited green fluorescence.

### Example 5

### Transgenic Soybean Containing a VP16ER Construct

Cotyledon and root explants from aseptically germinated soybean seedlings were cocultivated with Agrobacterium containing the Bin4-EUTG-10::VPCR-ER-1#81 vector. After 2 days, treated cotyledons and root explants were rinsed with liquid tissue culture medium to remove Agrobacterium and transferred to callus inducing medium (CIM) containing 0.5 mg/ml auxin, 0.5 mg/ml cytokinin, and 2.5 mg/ml bialaphos. More than 20 callus clusters that appeared to be surviving in the presence of bialaphos (putative transformants) were examined under a dissecting microscope at 10× to 60× magnification starting at 3 days after transfer to CIM. Several of the callus clusters examined exhibited moderate to strong green fluorescence. None of the explants from control plants transformed with the Bin4-EUTG-101 control construct exhibited green fluorescence.

### Example 6

### RXRα/VP16THRβ Nucleic Acid Construct

A T-DNA binary vector heterodimer nuclear receptor construct which encodes both RXRα and a chimeric VP16THRβ was prepared according to standard molecular biology techniques. The construct was designated Bin4-35S::RXRα-35S::VP16THRβ-3RXRE::Luc #12. The construct contained a 35S promoter operably linked to an RXRα coding sequence. See SEQ ID NOS: 5 and 6. The construct also contained a 35S promoter operably linked to a VP16THRβ coding sequence. See SEQ ID NOS: 7 and 8. The chimeric VP16THRβ receptor coding sequence included a VP16 activation domain coding sequence operably linked to a THRβ coding sequence. Each receptor coding sequence was operably linked to a 35S promoter. The binary vector also contained a reporter luciferase (Luc) coding sequence driven by a cis RXR-responsive element (RXRE) containing 3 repeats (see Table 3, where n=C), designated RXRE::Luc. See SEQ ID NOS: 9 and 10. A construct encoding the Luc coding sequence driven by 35S was also prepared and used for a positive control treatment, and a construct encoding a GFP coding sequence driven by 35S was prepared and used for a negative control treatment.

### Example 7

### Transient Transformation of Tobacco with a RXRα/VP16THRβ Construct

Plant tissue was transiently transformed as follows. A YEB culture (4 ml) of the Agrobacterium strain containing the Bin4-35S::RXRα-35S::VP16THRβ-3RXRE::Luc #12 construct of Example 6 was grown overnight at 28° C. with shaking. Agrobacterium was centrifuged at 4,000 rpm for 25 min and the bacterial pellet was resuspended in infiltration medium (MS solution plus 0.25 ug/ml NAA and 0.1 ug/ml BAP). A final 0.05-OD dilution of Agrobacterium was prepared using the same infiltration medium. Next, tobacco leaf discs were cut with a paper puncher and immediately immersed in infiltration medium. Leaf discs were then immersed in the diluted Agrobacterium culture for about 5 minutes, then blot-dried onto paper towels, before being transferred into a Petri dish lined with a paper towel that was pre-soaked with infiltration medium. Treated leaf discs were incubated for 4 days in a growth chamber maintained at 27° C., at a 16 hr light cycle.

Following incubation, leaf disks were subjected to treatment with exogenous ligands. Appropriate stocks of ligands were dissolved in DMSO, and diluted to a concentration of 5 uM using freshly made infiltration medium. Transiently-transformed leaf discs were then transferred into a Petri dish containing diluted ligand solution. The disks were then briefly rinsed with the ligand solution, and transferred into a new Petri dish lined with a paper towel pre-soaked with diluted ligand solution. Plates with either treated or control leaf discs were wrapped in aluminum foil and incubated for 1-2 days in a growth chamber set at 27° C. Following incubation, leaf discs were transferred onto plates overlaid with a thin layer of hardened 0.5% agarose. Leaf disks were next sprayed with a 10 uM luciferin (in 0.01% Triton X-100) preparation, and luminescent images were captured using a CCD imaging system. The results of these experiments are summarized in Table 4.

TABLE 4

| | Reporter expression |
|---|---|
| Ligand | in tobacco leaf disks |
| No Ligand | − |
| Triiodothyronine | ++ |
| Ciglitazone | − |
| Estradiol | − |
| Flutamide | +/− |
| 13-cis Retinoic Acid | +/− |
| 9-cis Retinoic Acid | − |

The results of these experiments indicate that binding of triiodothyronine to a chimeric VP16THRβ receptor occurs in tobacco, and that a chimeric VP16THRβ receptor functionally associates with an RXRα receptor to form a heterodimer, which in turn increases transcription of a reporter gene operably linked to an RXR response element.

### Example 8

### In Planta Testing of a RXRα/VP16THRβ Heterodimer Construct

Agrobacterium containing the Bin4-35S::RXRα-35S::VP16THR-3RXRE::Luc #12 construct of Example 6 was used to transiently transform Atropa belladonna, Digitalis purpurea (Fox Glove Purple), Oryza sativa (Japonica), Plantago lanceolata, Salvia coccinea (Spanish sage, Cat#1835, Lady in Red variety), Salvia coccinea (Spanish sage, Cat#1831, Cherry Blossom variety), and Withania somniferum. Seeds were obtained from Thompson & Morgan Seedsmen Inc. (Jackson, N.J.) and Sand Mountain Herbs (Fyffe, Ala.). Transient transformation was carried out similar to that described in Example 7. Following incubation, leaf disks of the different species were subjected to a treatment with the ligand triiodothyronine, as described in Example 7. Following incubation with triiodothyronine, leaf discs were transferred onto plates overlaid with a thin layer of hardened 0.5% agarose, and sprayed with a 10 uM luciferin (in 0.01% Triton X-100) preparation, and luminescent images were captured using a CCD imaging system. The results of these experiments are summarized in Table 5.

TABLE 5

| | | Reporter expression | Reporter |
|---|---|---|---|
| | | in the absence of | expression |
| Species | Code | exogenous ligand | (+Triiodothyronine) |
| Atropa belladona | Ab | − | ++ |
| Digitalis purpurea | Dp | +++ | + |
| Oryza sativa | Os | + | ++ |
| Plantago lanceolata | P1 | − | ++ |
| Salvia coccinea (cv. | Sc1835 | ++ | ++ |
| Lady in Red) | | | |
| Salvia coccinea (cv. | Sc1831 | − | ++ |
| Cherry Blossom) | | | |
| Withania | Ws | + | ++ |
| somniferum | | | |

The results of these experiments indicate that triiodothyronine binds to a chimeric VP16THRβ receptor in a variety of plant species, and that a chimeric VP16THRβ receptor functionally associates with an RXRα receptor to form a heterodimer, which in turn increases transcription of a reporter gene operably linked to an RXR response element. In Digitalis, Luc reporter activity was detected at a higher level in the absence of triiodothyronine and than in its presence. These results could be due to a negative effect that triiodothyronine may exert in Digitalis, or due to reduced reporter stability in this species. In the absence of triiodothyronine, Sc1835 showed reporter expression, whereas Sc1831 did not. This difference could be due to one or more candidate ligands that are present in one cultivar and absent in the other. Such a ligand may activate reporter gene transcription in the absence of an exogenous ligand.

### Example 9

### In Planta Testing of a VP16ER Homodimer Construct

A T-DNA binary vector nuclear receptor construct which encodes a chimeric ER hormone receptor was prepared according to standard molecular biology techniques. The construct was designated Bin4-35S::VP16ER-ERE::Luc #3. The chimeric VP16ER receptor coding sequence was operably linked to a 35S promoter. See SEQ ID NOS: 11 and 12. The binary vector also contained a Luc coding sequence driven by a cis ER-responsive element (ERE) containing 5 repeats. See Table 3, where nnn=GGG, and SEQ ID NOS: 13 and 14.

The Bin4-35S::VP16ER-ERE::Luc #3 construct was transiently transformed into a number of plant species essentially as described above. Seeds were obtained from Thompson & Morgan Seedsmen Inc. (Jackson, N.J.) and Sand Mountain Herbs (Fyffe, Ala.).

Control leaf disks were subjected to incubation in infiltration medium. Test leaf disks were subjected to incubation in infiltration medium, followed with incubation with estradiol. Leaf disks were next transferred onto plates overlaid with a thin layer of hardened 0.5% agarose, and sprayed with a 10 uM luciferin (in 0.01% Triton X-100) preparation. Luminescence images of both the control and test leaf were captured using a CCD imaging system. The results of these experiments are summarized in Table 6.

TABLE 6

| | | | Reporter |
|---|---|---|---|
| | | | expression |
| | | | in the |
| | | | absence of |
| | | | exogenous |
| Species | Variety name | Code | ligand |
| Arabidopsis thaliana | Wassilewskija | At | − |
| Agastache officinalis | Pink variety | Ao | − |
| Datura sp. | Double Blackcurrent Swirl | Db | + |
| Echinacea purpurea | White swan | Ep2727 | − |
| Echinacea purpurea | Magnus | Ep2733 | − |
| Eschscholtzia | Sun shades | Ec | + |
| californica | | | |
| Glycine max | Unknown | Gm | − |
| Hyssopus officinale | Blue variety | HoBlue | − |
| Ocimum basilicum | Basil Siam Queen | Ob0059 | − |
| | (Cat#0059) | | |
| Ocimum basilicum | Sweet basil Green | Ob0474 | − |
| | (Cat#0474) | | |
| Oenothera odorata | Evening primrose, Apricot | Oo6739 | + |
| | Delight (Cat#6739) | | |
| Oenothera odorata | Evening primrose, Pink | Oo8881 | ++ |
| | Petticoats (Cat#8881) | | |
| Plantago lanceolata | Unknown | P1 | − |
| Rheum palmatum | Cat#2142 | Rp2142 | + |
| Salvia coccinea | Spanish sage, Cherry | Sc1831 | − |
| | Blossom (Cat#1831) | | |
| Trifolium pratense | Unknown | Tp | − |
| Vitex negundo | Heterophylla | VnH | − |
| Withania somniferum | Unknown | Ws | +/− |

The results of these experiments suggest that Datura sp., Eschscholtzia californica, Oenothera odorata (Oo6739), Oenothera odorata (Oo8881), and Rheum palmatum possess candidate ligands that bind to estrogen nuclear receptor polypeptides. These results further suggest that such ligands can induce such polypeptides to activate transcription in plants.

### Example 10

### AR Nucleic Acid Construct

A T-DNA binary vector nuclear receptor construct, Bin4-Ins5ARE::Luc-35S::AR#2, which encodes an Androgen Receptor (AR) was prepared according to standard molecular biology techniques. The receptor coding sequence was operably linked to a 35S promoter (SEQ ID NOS: 15 and 16). The binary vector also contained a reporter luciferase (Luc) coding sequence driven by a cis Androgen Receptor-responsive element (ARE). See SEQ ID NOS: 17 and 18. The ARE contained 5 repeats of 5′-AGAACACTGTGTACC-3′ (SEQ ID NO: 19), operably linked to an insulator sequence.

### Example 11

### Transient Transformation of Tobacco with AR Construct

Plant tissue was transiently transformed essentially as described in Example 7, using the Agrobacterium strain containing the Bin4-Ins5ARE::Luc-35S::AR#2 construct of Example 10. The results of these experiments are summarized in Table 7.

TABLE 7

| | Reporter expression |
|---|---|
| Ligand | in tobacco leaf disks |
| No Ligand | − |
| Ciglitazone | +/− |
| Estradiol | +/− |
| Flutamide | ++ |
| Triiodothyronine | +/− |
| 9-cis Retinoic Acid | ++ |
| 13-cis Retinoic Acid | ++ |

The results of these experiments indicate that binding of flutamide, 9-cis retinoic acid, and 13-cis retinoic acid to an AR receptor occurs in tobacco.

### Example 12

### In Planta Testing of an AR Construct

Agrobacterium containing the Bin4-Ins5ARE::Luc-35S::AR#2 construct of Example 10 was used to transiently transform Atropa belladonna, Echinacea purpurea, Ocimum basilicum, Oenothera odorata, Oryza sativa, and Plantago lanceolata. Transient transformation was carried out in a manner similar to that described in Example 7. Following incubation, leaf disks of the different species were subjected to a treatment with the ligand flutamide, as described in Example 7. Following incubation with flutamide, leaf discs were transferred onto plates overlaid with a thin layer of hardened 0.5% agarose, and sprayed with a 10 uM luciferin (in 0.01% Triton X-100) preparation, and luminescent images were captured using a CCD imaging system. The results of these experiments are summarized in Table 8.

TABLE 8

| | | Reporter expression | Reporter |
|---|---|---|---|
| | | in the absence of | expression |
| Species | Code | exogenous ligand | (+flutamide) |
| Atropa belladona | Ab | +/− | + |
| Echinacea purpurea | Ep2727 | − | +/− |
| (cv. White swan) | | | |
| Ocimum basilicum (cv. | Ob0474 | + | ++ |
| Sweet basil Green) | | | |
| Oenothera odorata (cv. | Oo6739 | +/− | +/− |
| Apricot delight) | | | |
| Oryza sativa | Os | + | + |
| Plantago lanceolata | P1 | +/− | + |

The results of these experiments suggest that Ocimum basilicum and Oryza sativa possess candidate ligands that bind to androgen nuclear receptor polypeptides. These results further suggest that such ligands can induce such polypeptides to activate transcription in plants. The results further suggest that addition of flutamide to the transgenic Plantago lanceolata activated reporter gene transcription.

### Example 13

### Identification of Endogenous Ligands having Estrogen Receptor Ligand Activity

Two-hundred and fifty mg of California poppy callus of Example 4 was freeze-dried and subjected to extraction in methanol using a Dionex ASE® 200 Accelerated Solvent Extractor (Sunnyvale, Calif.). Crude extracts were dried and resuspended in pure methanol. Resuspended crude extract was run in a semi-prep HPLC gradient of 20% to 95% acetonitrile in 0.1% formic acid for 55 min using an Alltima 150×10 mm-Sum C18 column using a Dionex Summit® HPLC system and fractionated at 4.7 ml/fraction. Eluted fractions were dried under vacuum and resuspended in 100 ul of DMSO/fraction. In order to determine which fractions had ER ligand activity, fractions were used to carry out in vitro assays using the HitHunter Estrogen kit (Discoverx, Fremont, Calif.) and pure Estrogen Receptor protein (Panvera/Invitrogen, Carlsbad, Calif.). Two ul aliquots of each fraction were used for the assay, and each assay was performed in three replicates. The results indicated that several fractions possessed ER ligand activity.

In order to determine the identity of compounds within active fractions, a crude extract of California poppy callus was prepared and resuspended in methanol as described above. Liquid chromatography fractionation and mass spectrometry (LC-MS) were carried out on the crude extract using a Waters Micromass® ZQ™ Mass Spectrometer (single quadrupole, benchtop MS detector; Milford, Mass.). The LC gradient was the same as that used for the semi-prep HPLC fractionation described above, using an Alltima 150×4.6 mm-Sum C18 column. Three of the peaks were identified as sanguinarine derivatives. These results demonstrate that plant cells expressing a nuclear hormone receptor polypeptide can be used to screen for candidate ligands, and that ligands that bind to such a receptor can be identified.

### Other Embodiments

The foregoing description is intended to illustrate and not limit the scope of the invention, which is defined by the scope of the appended claims. Other aspects, advantages, and modifications are within the scope of the following claims.

1. A method for identifying a ligand for a nuclear hormone receptor polypeptide, said method comprising:
a) providing a plurality of plant cells comprising a nucleic acid encoding a heterologous nuclear hormone receptor polypeptide; and
b) determining whether one or more candidate ligands interact with said receptor using a reporter responsive to signal transduction activity of said receptor.

2. The method of claim 1, wherein said plant cells are a part of at least one whole plant.

3. The method of claim 1, wherein said plant cells are cells in tissue culture.

4. The method of claim 3, wherein said tissue culture is a callus culture.

5. The method of claim 1, wherein said heterologous receptor is a mammalian nuclear hormone receptor.

6. The method of claim 5, wherein said heterologous receptor is selected from the group consisting of AR, RAR, ROR, LRH-1, THR, VDR, PPAR, LXR, ER, CAR, FXR, and RXR.

7. The method of claim 1, wherein said plurality of plant cells further comprise a nucleic acid encoding a dimerization receptor polypeptide.

8. The method of claim 1, wherein said heterologous receptor is a chimeric receptor.

9. The method of claim 8, wherein said heterologous receptor comprises a first segment, a second segment, and a third segment.

10. The method of claim 9, wherein said first segment comprises a ligand binding domain, said second segment comprises a DNA binding domain, and said third segment comprises a transactivation domain.

11. The method of claim 10, wherein said transactivation domain of said chimeric receptor is a VP16 transactivation domain or a maize transcription factor C transactivation domain.

12. The method of claim 10, wherein said DNA binding domain of said chimeric receptor is a DNA binding domain of AR, RAR, ROR, LRH-1, THR, VDR, PPAR, LXR, ER, CAR, FXR, or RXR.

13. The method of claim 1, wherein said one or more candidate ligands are synthesized by said plant cells.

14. The method of claim 1, wherein said reporter is a polypeptide having a spectrophotometrically measurable activity.

15. The method of claim 14, wherein said spectrophotometrically measurable activity is fluorescence or bioluminescence.

16. The method of claim 14, wherein said reporter comprises an epitope tag.

17. The method of claim 1, further comprising isolating cells that express said reporter.

18. The method of claim 1, further comprising using mass spectroscopy to identify said ligand.

19. The method of claim 1, wherein said nucleic acid is operably linked to a regulatory element is capable of conferring expression in plant cells.

20. The method of claim 1, wherein said plurality of plant cells further comprise a nucleic acid encoding a polypeptide selected from the group consisting of a terpenoid biosynthesis polypeptide, a flavonoid biosynthesis polypeptide, a phenolic biosynthesis polypeptide and an alkaloid biosynthesis polypeptide.

21. A method of screening for a nuclear hormone receptor ligand, comprising:
a) providing one or more plant cells comprising at least one construct, said construct comprising a coding sequence for a nuclear hormone receptor polypeptide and a sequence to be transcribed as a reporter,
wherein said nuclear hormone receptor polypeptide comprises a ligand binding domain, a DNA binding domain, and a transactivation domain, wherein said sequence to be transcribed as a reporter is operably linked to a receptor response element capable of interacting with said DNA binding domain, and wherein activity of said reporter is detectable upon receptor/ligand binding-dependent transcription of said sequence;
b) permitting a candidate ligand to contact said receptor polypeptide under conditions that allow transcription of said receptor polypeptide from said construct and binding of said ligand thereto; and
c) determining whether activity of said reporter is detected.

22. The method of claim 21, wherein said plant cells are a part of at least one whole plant.

23. The method of claim 21, wherein said plant cells are cells in tissue culture.

24. The method of claim 23, wherein said tissue culture is a callus culture.

25. The method of claim 21, wherein said nuclear hormone receptor is a mammalian nuclear hormone receptor.

26. The method of claim 25, wherein said nuclear hormone receptor is selected from the group consisting of AR, RAR, ROR, LRH-1, THR, VDR, PPAR, LXR, ER, CAR, FXR, and RXR.

27. The method of claim 21, wherein said nuclear hormone receptor is a chimeric receptor.

28. The method of claim 27, wherein said transactivation domain of said chimeric receptor is a VP16 transactivation domain or a maize transcription factor C transactivation domain.

29. The method of claim 27, wherein said DNA binding domain of said chimeric receptor is a DNA binding domain of AR, RAR, ROR, LRH-1, THR, VDR, PPAR, LXR, ER, CAR, FXR, or RXR.

30. The method of claim 21, wherein said one or more candidate ligands are synthesized by said plant cells.

31. The method of claim 21, wherein said sequence to be transcribed as a reporter encodes a polypeptide having a spectrophotometrically measurable activity.

32. The method of claim 31, wherein said sequence to be transcribed as a reporter further encodes an epitope tag.

33. The method of claim 31, wherein said spectrophotometrically measurable activity is fluorescence or bioluminescence.

34. The method of claim 21, wherein said coding sequence for a nuclear hormone receptor polypeptide is operably linked to a regulatory element conferring constitutive expression

35. The method of claim 21, further comprising using mass spectroscopy to identify said ligand.

36. The method of claim 21, further comprising isolating cells that express said reporter.

37. A transgenic plant cell comprising a first recombinant nucleic acid, said first recombinant nucleic acid comprising a regulatory element operably linked to a coding sequence for a polypeptide having greater than 40% sequence identity to a nuclear hormone receptor polypeptide, and a second recombinant nucleic acid comprising a nuclear receptor response element operably linked to a reporter coding sequence.

38. The transgenic plant cell of claim 37, wherein said nuclear hormone receptor polypeptide is selected from the group consisting of AR, RAR, ROR, LRH-1, THR, VDR, PPAR, LXR, ER, CAR, FXR, or RXR.

39. The transgenic plant cell of claim 37, wherein plant cell is a transiently transformed plant cell.

40. The transgenic plant cell of claim 37, wherein said plant cell is a part of a whole plant.

41. The transgenic plant cell of claim 37, wherein said plant cell is a cell in tissue culture.


SEQUENCE LISTING (48 sequences)

SEQ ID NO: 1 - DNA, 1793 bp, Artificial Sequence
  synthetically generated cassette
atgacggcccccccgaccgacgtcagcctgggggacgagctccacttagacggcgaggacgtggcgatggcgcatgccgacgcgctagacgatttcgatctggacatgttgggggacggggattccccggggccgggatttaccccccacgactccgccccctacggcgctctggatacggccgacttcgagtttgagcagatgtttaccgatgcccttggaattgacgagtacggtgggtccaacggcctggggggtttccccccactcaacagcgtgtctccgagcccgctgatgctactgcacccgccgccgcagctgtcgcctttcctgcagccccacggccagcaggtgccctactacctggagaacgagcccagcggctacacggtgcgcgaggccggcccgccggcattctacaggccaaattcagataatcgacgccagggtggcagagaaagattggccagtaccaatgacaagggaagtatggctatggaatctgccaaggagactcgctactgtgcagtgtgcaatgactatgcttcaggctaccattatggagtctggtcctgtgagggctgcaaggccttcttcaagagaagtattcaaggacataacgactatatgtgtccagccaccaaccagtgcaccattgataaaaacaggaggaagagctgccaggcctgccggctccgcaaatgctacgaagtgggaatgatgaaaggtgggatacgaaaagaccgaagaggagggagaatgttgaaacacaagcgccagagagatgatggggagggcaggggtgaagtggggtctgctggagacatgagagctgccaacctttggccaagcccgctcatgatcaaacgctctaagaagaacagcctggccttgtccctgacggccgaccagatggtcagtgccttgttggatgctgagccccccatactctattccgagtatgatcctaccagacccttcagtgaagcttcgatgatgggcttactgaccaacctggcagacagggagctggttcacatgatcaactgggcgaagagggtgccaggctttgtggatttgaccctccatgatcaggtccaccttctagaatgtgcctggctagagatcctgatgattggtctcgtctggcgctccatggagcacccagtgaagctactgtttgctcctaacttgctcttggacaggaaccagggaaaatgtgtagagggcatggtggagatcttcgacatgctgctggctacatcatctcggttccgcatgatgaatctgcagggagaggagtttgtgtgcctcaaatctattattttgcttaattctggagtgtacacatttctgtccagcaccctgaagtctctggaagagaaggaccatatccaccgagtcctggacaagatcacagacactttgatccacctgatggccaaggcaggcctgaccctgcagcagcagcaccagcggctggcccagctcctcctcatcctctcccacatcaggcacatgagtaacaaaggcatggagcatctgtacagcatgaagtgcaagaacgtggtgcccctctatgacctgctgctggagatgctggacgcccaccgcctacatgcgcccactagccgtggaggggcatccgtggaggagacggaccaaagccacttggccactgcgggctctacttcatcgcattccttgcaaaagtattacatcacgggggaggcagagggtttccctgccacagtctgagagctccctggctcccacacggttcagataatccc

SEQ ID NO: 2 - protein, 585 aa, Artificial Sequence
  synthetically generated peptide
MTAPPTDVSLGDELHLDGEDVAMAHADALDDFDLDMLGDGDSPGPGFTPHDSAPYGALDTADFEFEQMFTDALGIDEYGGSNGLGGFPPLNSVSPSPLMLLHPPPQLSPFLQPHGQQVPYYLENEPSGYTVREAGPPAFYRPNSDNRRQGGRERLASTNDKGSMAMESAKETRYCAVCNDYASGYHYGVWSCEGCKAFFKRSIQGHNDYMCPATNQCTIDKNRRKSCQACRLRKCYEVGMMKGGIRKDRRGGRMLKHKRQRDDGEGRGEVGSAGDMRAANLWPSPLMIKRSKKNSLALSLTADQMVSALLDAEPPILYSEYDPTRPFSEASMMGLLTNLADRELVHMINWAKRVPGFVDLTLHDQVHLLECAWLEILMIGLVWRSMEHPVKLLFAPNLLLDRNQGKCVEGMVEIFDMLLATSSRFRMMNLQGEEFVCLKSIILLNSGVYTFLSSTLKSLEEKDHIHRVLDKITDTLIHLMAKAGLTLQQQHQRLAQLLLILSHIRHMSNKGMEHLYSMKCKNVVPLYDLLLEMLDAHRLHAPTSRGGASVEETDQSHLATAGSTSSHSLQKYYITGEAEGFPATV

SEQ ID NO: 3 - DNA, 1434 bp, Artificial Sequence
  synthetically generated cassette
cagggtcaccctgacctgagctactcgaccaggtcaccctgacctgagctactcgaccaggtcaccctgacctgctcgagcaggtcaccctgacctgagctactcgaccaggtcaccctgacctgagctactcgatcaccctgacctggatccaaggagggccatagcggccgcggaggactgaggactgtcctccgttcggaggactgtcctccgaccggaggactgtcctcggaggactgtcctccgctttctcgtgacctagtcgtccttgtcttttcttcttcttcttctataaaacaatacccaaagagctccaccgcggtggcggccggccgctctagaactagtggatccaaggagatataacaatgaagactaatctttttctctttctcatcttttcacttctcctatcattatcctcggccgaattcagtaaaggagaagaacttttcactggagttgtcccaattcttgttgaattagatggtgatgttaatgggcacaaattttctgtcagtggagagggtgaaggtgatgcaacatacggaaaacttacccttaaatttatttgcactactggaaaactacctgttccatggccaacacttgtcactactttctcttatggtgttcaatgcttttcaagatacccagatcatatgaagcggcacgacttcttcaagagcgccatgcctgagggatacgtgcaggagaggaccatcttcttcaaggacgacgggaactacaagacacgtgctgaagtcaagtttgagggagacaccctcgtcaacaggatcgagcttaagggaatcgatttcaaggaggacggaaacatcctcggccacaagttggaatacaactacaactcccacaacgtatacatcatggccgacaagcaaaagaacggcatcaaagccaacttcaagacccgccacaacatcgaagacggcggcgtgcaactcgctgatcattatcaacaaaatactccaattggcgatggccctgtccttttaccagacaaccattacctgtccacacaatctgccctttcgaaagatcccaacgaaaagagagaccacatggtccttcttgagtttgtaacagctgctgggattacacatggcatggatgaactatacaaacatgatgagctttaagagctcgaatttccccgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgg

SEQ ID NO: 4 - protein, 263 aa, Aequorea victoria
MKTNLFLFLIFSLLLSLSSAEFSKGEELFTGVVPILVELDGDVNGHKFSVSGEGEGDATYGKLTLKFICTTGKLPVPWPTLVTTFSYGVQCFSRYPDHMKRHDFFKSAMPEGYVQERTIFFKDDGNYKTRAEVKFEGDTLVNRIELKGIDFKEDGNILGHKLEYNYNSHNVYIMADKQKNGIKANFKTRHNIEDGGVQLADHYQQNTPIGDGPVLLPDNHYLSTQSALSKDPNEKRDHMVLLEFVTAAGITHGMDELYKHDEL

SEQ ID NO: 5 - DNA, 1443 bp, Artificial Sequence
  synthetically generated cassette
gcatgagttagtcgcagacatggacaccaaacatttcctgccgctcgatttctccacccaggtgaactcctccctcacctccccgacggggcgaggctccatggctgccccctcgctgcacccgtccctggggcctggcatcggctccccgggacagctgcattctcccatcagcaccctgagctcccccatcaacggcatgggcccgcctttctcggtcatcagctcccccatgggcccccactccatgtcggtgcccaccacacccaccctgggcttcagcactggcagcccccagctcagctcacctatgaaccccgtcagcagcagcgaggacatcaagccccccctgggcctcaatggcgtcctcaaggtccccgcccacccctcaggaaacatggcttccttcaccaagcacatctgcgccatctgcggggaccgctcctcaggcaagcactatggagtgtacagctgcgaggggtgcaagggcttcttcaagcggacggtgcgcaaggacctgacctacacctgccgcgacaacaaggactgcctgattgacaagcggcagcggaaccggtgccagtactgccgctaccagaagtgcctggccatgggcatgaagcgggaagccgtgcaggaggagcggcagcgtggcaaggaccggaacgagaatgaggtggagtcgaccagcagcgccaacgaggacatgccggtggagaggatcctggaggctgagctggccgtggagcccaagaccgagacctacgtggaggcaaacatggggctgaaccccagctcgccgaacgaccctgtcaccaacatttgccaagcagccgacaaacagcttttcaccctggtggagtgggccaagcggatcccacacttctcagagctgcccctggacgaccaggtcatcctgctgcgggcaggctggaatgagctgctcatcgcctccttctcccaccgctccatcgccgtgaaggacgggatcctcctggccaccgggctgcacgtccaccggaacagcgcccacagcgcaggggtgggcgccatctttgacagggtgctgacggagcttgtgtccaagatgcgggacatgcagatggacaagacggagctgggctgcctgcgcgccatcgtcctctttaaccctgactccaaggggctctcgaacccggccgaggtggaggcgctgagggagaaggtctatgcgtccttggaggcctactgcaagcacaagtacccagagcagccgggaaggttcgctaagctcttgctccgcctgccggctctgcgctccatcgggctcaaatgcctggaacatctcttcttcttcaagctcatcggggacacacccattgacaccttccttatggagatgctggaggcgccgcaccaaatgacttaggcctgcgggcccatcctttgtgcccacccgttctg

SEQ ID NO: 6 - protein, 462 aa, Artificial Sequence
  synthetically generated peptide
MDTKHFLPLDFSTQVNSSLTSPTGRGSMAAPSLHPSLGPGIGSPGQLHSPISTLSSPINGMGPPFSVISSPMGPHSMSVPTTPTLGFSTGSPQLSSPMNPVSSSEDIKPPLGLNGVLKVPAHPSGNMASFTKHICAICGDRSSGKHYGVYSCEGCKGFFKRTVRKDLTYTCRDNKDCLIDKRQRNRCQYCRYQKCLAMGMKREAVQEERQRGKDRNENEVESTSSANEDMPVERILEAELAVEPKTETYVEANMGLNPSSPNDPVTNICQAADKQLFTLVEWAKRIPHFSELPLDDQVILLRAGWNELLIASFSHRSIAVKDGILLATGLHVHRNSAHSAGVGAIFDRVLTELVSKMRDMQMDKTELGCLRAIVLFNPDSKGLSNPAEVEALREKVYASLEAYCKHKYPEQPGRFAKLLLRLPALRSIGLKCLEHLFFFKLIGDTPIDTFLMEMLEAPHQMT

SEQ ID NO: 7 - DNA, 1641 bp, Artificial Sequence
  synthetically generated cassette
ccagtgtgctggaacaatgacggcccccccgaccgacgtcagcctgggggacgagctccacttagacggcgaggacgtggcgatggcgcatgccgacgcgctagacgatttcgatctggacatgttgggggacggggattccccggggccgggatttaccccccacgactccgccccctacggcgctctggatacggccgacttcgagtttgagcagatgtttaccgatgcccttggaattgacgagtacggtgggtccaacggcctggaagggcgaattccagcacactggcggccgttactagtggatccgctagtaggaatgtctgaagcctgcctacataggaagagccattcagagaggcgcagcacgttgaaaaatgaacagtcgtcgccacatctcatccagaccacttggactagctcaatattccatctggaccatgatgatgtgaacgaccagagtgtctcaagtgcccagaccttccaaacggaggagaagaaatgtaaagggtacatccccagttacttagacaaggacgagctctgtgtagtgtgtggtgacaaagccaccgggtatcactaccgctgtatcacgtgtgaaggctgcaagggtttctttagaagaaccattcagaaaaatctccatccatcctattcctgtaaatatgaaggaaaatgtgtcatagacaaagtcacgcgaaatcagtgccaggaatgtcgctttaagaaatgcatctatgttggcatggcaacagatttggtgctggatgacagcaagaggctggccaagaggaagctgatagaggagaaccgggagaaaagacggcgggaagagctgcagaagtccatcgggcacaagccagagcccacagacgaggaatgggagctcatcaaaactgtcaccgaagcccatgtggcgaccaacgcccaaggcagccactggaagcaaaaacggaaattcctgccagaagacattggacaagcaccaatagtcaatgccccagaaggtggaaaggttgacttggaagccttcagccattttacaaaaatcatcacaccagcaattaccagagtggtggattttgccaaaaagttgcctatgttttgtgagctgccatgtgaagaccagatcatcctcctcaaaggctgctgcatggagatcatgtcccttcgcgctgctgtgcgctatgacccagaaagtgagactttaaccttgaatggggaaatggcagtgacacggggccagctgaaaaatgggggtcttggggtggtgtcagacgccatctttgacctgggcatgtctctgtcttctttcaacctggatgacactgaagtagccctccttcaggccgtcctgctgatgtcttcagatcgcccggggcttgcctgtgttgagagaatagaaaagtaccaagatagtttcctgctggcctttgaacactatatcaattaccgaaaacaccacgtgacacacttttggccaaaactcctgatgaaggtgacagatctgcggatgataggagcctgccatgccagccgcttcctgcacatgaaggtggaatgccccacagaactcttcccccctttgttcttggaagtgttcgaggattagactgactggattcattctcataattc

SEQ ID NO: 8 - protein, 532 aa, Artificial Sequence
  synthetically generated peptide
MTAPPTDVSLGDELHLDGEDVAMAHADALDDFDLDMLGDGDSPGPGFTPHDSAPYGALDTADFEFEQMFTDALGIDEYGGSNGLEGRIPAHWRPLLVDPLVGMSEACLHRKSHSERRSTLKNEQSSPHLIQTTWTSSIFHLDHDDVNDQSVSSAQTFQTEEKKCKGYIPSYLDKDELCVVCGDKATGYHYRCITCEGCKGFFRRTIQKNLHPSYSCKYEGKCVIDKVTRNQCQECRFKKCIYVGMATDLVLDDSKRLAKRKLIEENREKRRREELQKSIGHKPEPTDEEWELIKTVTEAHVATNAQGSHWKQKRKFLPEDIGQAPIVNAPEGGKVDLEAFSHFTKIITPAITRVVDFAKKLPMFCELPCEDQIILLKGCCMEIMSLRAAVRYDPESETLTLNGEMAVTRGQLKNGGLGVVSDAIFDLGMSLSSFNLDDTEVALLQAVLLMSSDRPGLACVERIEKYQDSFLLAFEHYINYRKHHVTHFWPKLLMKVTDLRMIGACHASRFLHMKVECPTELFPPLFLEVFED

SEQ ID NO: 9 - DNA, 1812 bp, Artificial Sequence
  synthetically generated cassette
aggtcacaggtcagtcgagtagctaggtcacaggtcagtcgaggctctagacaggtcacaggtcaacctagtcgtcctcgtcttttcttcttcttcttctataaaacaatacccaaagagctggcttgacgaaggcattccggtactgttggtaaagccatggaagacgccaaaaacataaagaaaggcccggcgccattctatccgctggaagatggaaccgctggagagcaactgcataaggctatgaagagatacgccctggttcctggaacaattgcttttacagatgcacatatcgaggtggacatcacttacgctgagtacttcgaaatgtccgttcggttggcagaagctatgaaacgatatgggctgaatacaaatcacagaatcgtcgtatgcagtgaaaactctcttcaattctttatgccggtgttgggcgcgttatttatcggagttgcagttgcgcccgcgaacgacatttataatgaacgtgaattgctcaacagtatgggcatttcgcagcctaccgtggtgttcgtttccaaaaaggggttgcaaaaaattttgaacgtgcaaaaaaagctcccaatcatccaaaaaattattatcatggattctaaaacggattaccagggatttcagtcgatgtacacgttcgtcacatctcatctacctcccggttttaatgaatacgattttgtgccagagtccttcgatagggacaagacaattgcactgatcatgaactcctctggatctactggtctgcctaaaggtgtcgctctgcctcatagaactgcctgcgtgagattctcgcatgccagagatcctatttttggcaatcaaatcattccggatactgcgattttaagtgttgttccattccatcacggttttggaatgtttactacactcggatatttgatatgtggatttcgagtcgtcttaatgtatagatttgaagaagagctgtttctgaggagccttcaggattacaagattcaaagtgcgctgctggtgccaaccctattctccttcttcgccaaaagcactctgattgacaaatacgatttatctaatttacacgaaattgcttctggtggcgctcccctctctaaggaagtcggggaagcggttgccaagaggttccatctgccaggtatcaggcaaggatatgggctcactgagactacatcagctattctgattacacccgagggggatgataaaccgggcgcggtcggtaaagttgttccattttttgaagcgaaggttgtggatctggataccgggaaaacgctgggcgttaatcaaagaggcgaactgtgtgtgagaggtcctatgattatgtccggttatgtaaacaatccggaagcgaccaacgccttgattgacaaggatggatggctacattctggagacatagcttactgggacgaagacgaacacttcttcatcgttgaccgcctgaagtctctgattaagtacaaaggctatcaggtggctcccgctgaattggaatccatcttgctccaacaccccaacatcttcgacgcaggtgtcgcaggtcttcccgacgatgacgccggtgaacttcccgccgccgttgttgttttggagcacggaaagacgatgacggaaaaagagatcgtggattacgtcgccagtcaagtaacaaccgcgaaaaagttgcgcggaggagttgtgtttgtggacgaagtaccgaaaggtcttaccggaaaactcgacgcaagaaaaatcagagagatcctcataaaggccaagaagggcggaaagatcgccgtgtaa

SEQ ID NO: 10 - protein, 550 aa, Artificial Sequence
  synthetically generated peptide
MEDAKNIKKGPAPFYPLEDGTAGEQLHKAMKRYALVPGTIAFTDAHIEVDITYAEYFEMSVRLAEAMKRYGLNTNHRIVVCSENSLQFFMPVLGALFIGVAVAPANDIYNERELLNSMGISQPTVVFVSKKGLQKILNVQKKLPIIQKIIIMDSKTDYQGFQSMYTFVTSHLPPGFNEYDFVPESFDRDKTIALIMNSSGSTGLPKGVALPHRTACVRFSHARDPIFGNQIIPDTAILSVVPFHHGFGMFTTLGYLICGFRVVLMYRFEEELFLRSLQDYKIQSALLVPTLFSFFAKSTLIDKYDLSNLHEIASGGAPLSKEVGEAVAKRFHLPGIRQGYGLTETTSAILITPEGDDKPGAVGKVVPFFEAKVVDLDTGKTLGVNQRGELCVRGPMIMSGYVNNPEATNALIDKDGWLHSGDIAYWDEDEHFFIVDRLKSLIKYKGYQVAPAELESILLQHPNIFDAGVAGLPDDDAGELPAAVVVLEHGKTMTEKEIVDYVASQVTTAKKLRGGVVFVDEVPKGLTGKLDARKIREILIKAKKGGKIAV

SEQ ID NO: 11 - DNA, 1809 bp, Artificial Sequence
  synthetically generated cassette
ccagtgtgctggaacaatgacggcccccccgaccgacgtcagcctgggggacgagctccacttagacggcgaggacgtggcgatggcgcatgccgacgcgctagacgatttcgatctggacatgttgggggacggggattccccggggccgggatttaccccccacgactccgccccctacggcgctctggatacggccgacttcgagtttgagcagatgtttaccgatgcccttggaattgacgagtacggtgggtccaacggcctggggggtttccccccactcaacagcgtgtctccgagcccgctgatgctactgcacccgccgccgcagctgtcgcctttcctgcagccccacggccagcaggtgccctactacctggagaacgagcccagcggctacacggtgcgcgaggccggcccgccggcattctacaggccaaattcagataatcgacgccagggtggcagagaaagattggccagtaccaatgacaagggaagtatggctatggaatctgccaaggagactcgctactgtgcagtgtgcaatgactatgcttcaggctaccattatggagtctggtcctgtgagggctgcaaggccttcttcaagagaagtattcaaggacataacgactatatgtgtccagccaccaaccagtgcaccattgataaaaacaggaggaagagctgccaggcctgccggctccgcaaatgctacgaagtgggaatgatgaaaggtgggatacgaaaagaccgaagaggagggagaatgttgaaacacaagcgccagagagatgatggggagggcaggggtgaagtggggtctgctggagacatgagagctgccaacctttggccaagcccgctcatgatcaaacgctctaagaagaacagcctggccttgtccctgacggccgaccagatggtcagtgccttgttggatgctgagccccccatactctattccgagtatgatcctaccagacccttcagtgaagcttcgatgatgggcttactgaccaacctggcagacagggagctggttcacatgatcaactgggcgaagagggtgccaggctttgtggatttgaccctccatgatcaggtccaccttctagaatgtgcctggctagagatcctgatgattggtctcgtctggcgctccatggagcacccagtgaagctactgtttgctcctaacttgctcttggacaggaaccagggaaaatgtgtagagggcatggtggagatcttcgacatgctgctggctacatcatctcggttccgcatgatgaatctgcagggagaggagtttgtgtgcctcaaatctattattttgcttaattctggagtgtacacatttctgtccagcaccctgaagtctctggaagagaaggaccatatccaccgagtcctggacaagatcacagacactttgatccacctgatggccaaggcaggcctgaccctgcagcagcagcaccagcggctggcccagctcctcctcatcctctcccacatcaggcacatgagtaacaaaggcatggagcatctgtacagcatgaagtgcaagaacgtggtgcccctctatgacctgctgctggagatgctggacgcccaccgcctacatgcgcccactagccgtggaggggcatccgtggaggagacggaccaaagccacttggccactgcgggctctacttcatcgcattccttgcaaaagtattacatcacgggggaggcagagggtttccctgccacagtctgagagctccctggctcccacacggttcagataatccc

SEQ ID NO: 12 - protein, 585 aa, Artificial Sequence; identical to SEQ ID NO: 2
  synthetically generated peptide

SEQ ID NO: 13 - DNA, 1999 bp, Artificial Sequence
  synthetically generated cassette
cagggtcaccctgacctgagctactcgaccaggtcaccctgacctgagctactcgaccaggtcaccctgacctgctcgagcaggtcaccctgacctgagctactcgaccaggtcaccctgacctgagctactcgatcaccctgacctggatccaaggagggccatagcggccgcggaggactgaggactgtcctccgttcggaggactgtcctccgaccggaggactgtcctcggaggactgtcctccgctttctcgtgacctagtcgtccttgtcttttcttcttcttcttctataaaacaatacccaaagagcttggcattccggtactgttggtaaagccaccatggaagacgccaaaaacataaagaaaggcccggcgccattctatccgctggaagatggaaccgctggagagcaactgcataaggctatgaagagatacgccctggttcctggaacaattgcttttacagatgcacatatcgaggtggacatcacttacgctgagtacttcgaaatgtccgttcggttggcagaagctatgaaacgatatgggctgaatacaaatcacagaatcgtcgtatgcagtgaaaactctcttcaattctttatgccggtgttgggcgcgttatttatcggagttgcagttgcgcccgcgaacgacatttataatgaacgtgaattgctcaacagtatgggcatttcgcagcctaccgtggtgttcgtttccaaaaaggggttgcaaaaaattttgaacgtgcaaaaaaagctcccaatcatccaaaaaattattatcatggattctaaaacggattaccagggatttcagtcgatgtacacgttcgtcacatctcatctacctcccggttttaatgaatacgattttgtgccagagtccttcgatagggacaagacaattgcactgatcatgaactcctctggatctactggtctgcctaaaggtgtcgctctgcctcatagaactgcctgcgtgagattctcgcatgccagagatcctatttttggcaatcaaatcattccggatactgcgattttaagtgttgttccattccatcacggttttggaatgtttactacactcggatatttgatatgtggatttcgagtcgtcttaatgtatagatttgaagaagagctgtttctgaggagccttcaggattacaagattcaaagtgcgctgctggtgccaaccctattctccttcttcgccaaaagcactctgattgacaaatacgatttatctaatttacacgaaattgcttctggtggcgctcccctctctaaggaagtcggggaagcggttgccaagaggttccatctgccaggtatcaggcaaggatatgggctcactgagactacatcagctattctgattacacccgagggggatgataaaccgggcgcggtcggtaaagttgttccattttttgaagcgaaggttgtggatctggataccgggaaaacgctgggcgttaatcaaagaggcgaactgtgtgtgagaggtcctatgattatgtccggttatgtaaacaatccggaagcgaccaacgccttgattgacaaggatggatggctacattctggagacatagcttactgggacgaagacgaacacttcttcatcgttgaccgcctgaagtctctgattaagtacaaaggctatcaggtggctcccgctgaattggaatccatcttgctccaacaccccaacatcttcgacgcaggtgtcgcaggtcttcccgacgatgacgccggtgaacttcccgccgccgttgttgttttggagcacggaaagacgatgacggaaaaagagatcgtggattacgtcgccagtcaagtaacaaccgcgaaaaagttgcgcggaggagttgtgtttgtggacgaagtaccgaaaggtcttaccggaaaactcgacgcaagaaaaatcagagagatcctcataaaggccaagaagggcggaaagatcgccgtgtaa

SEQ ID NO: 14 - protein, 550 aa, Artificial Sequence; identical to SEQ ID NO: 10
  synthetically generated peptide

SEQ ID NO: 15 - DNA, 2861 bp, Artificial Sequence
  synthetically generated cassette
cctgttgaactcttctgagcaagagaaggggaggcggggtaagggaagtaggtggaagattcagccaagctcaaggatggaagtgcagttagggctgggaagggtctaccctcggccgccgtccaagacctaccgaggagctttccagaatctgttccagagcgtgcgcgaagtgatccagaacccgggccccaggcacccagaggccgcgagcgcagcacctcccggcgccagtttgctgctgctgcagcagcagcagcagcagcagcagcagcagcagcagcagcagcagcagcagcagcagcagcagcagcaagagactagccccaggcagcagcagcagcagcagggtgaggatggttctccccaagcccatcgtagaggccccacaggctacctggtcctggatgaggaacagcaaccttcacagccgcagtcggccctggagtgccaccccgagagaggttgcgtcccagagcctggagccgccgtggccgccagcaaggggctgccgcagcagctgccagcacctccggacgaggatgactcagctgccccatccacgttgtccctgctgggccccactttccccggcttaagcagctgctccgctgaccttaaagacatcctgagcgaggccagcaccatgcaactccttcagcaacagcagcaggaagcagtatccgaaggcagcagcagcgggagagcgagggaggcctcgggggctcccacttcctccaaggacaattacttagggggcacttcgaccatttctgacaacgccaaggagttgtgtaaggcagtgtcggtgtccatgggcctgggtgtggaggcgttggagcatctgagtccaggggaacagcttcggggggattgcatgtacgccccacttttgggagttccacccgctgtgcgtcccactccttgtgccccattggccgaatgcaaaggttctctgctagacgacagcgcaggcaagagcactgaagatactgctgagtattcccctttcaagggaggttacaccaaagggctagaaggcgagagcctaggctgctctggcagcgctgcagcagggagctccgggacacttgaactgccgtctaccctgtctctctacaagtccggagcactggacgaggcagctgcgtaccagagtcgcgactactacaactttccactggctctggccggaccgccgccccctccgccgcctccccatccccacgctcgcatcaagctggagaacccgctggactacggcagcgcctgggcggctgcggcggcgcagtgccgctatggggacctggcgagcctgcatggcgcgggtgcagcgggacccggttctgggtcaccctcagccgccgcttcctcatcctggcacactctcttcacagccgaagaaggccagttgtatggaccgtgtggtggtggtgggggtggtggcggcggcggcggcggcggcggcggcggcggcggcggcggcggcggcggcgaggcgggagctgtagccccctacggctacactcggccccctcaggggctggcgggccaggaaagcgacttcaccgcacctgatgtgtggtaccctggcggcatggtgagcagagtgccctatcccagtcccacttgtgtcaaaagcgaaatgggcccctggatggatagctactccggaccttacggggacatgcgtttggagactgccagggaccatgttttgcccattgactattactttccaccccagaagacctgcctgatctgtggagatgaagcttctgggtgtcactatggagctctcacatgtggaagctgcaaggtcttcttcaaaagagccgctgaagggaaacagaagtacctgtgcgccagcagaaatgattgcactattgataaattccgaaggaaaaattgtccatcttgtcgtcttcggaaatgttatgaagcagggatgactctgggagcccggaagctgaagaaacttggtaatctgaaactacaggaggaaggagaggcttccagcaccaccagccccactgaggagacaacccagaagctgacagtgtcacacattgaaggctatgaatgtcagcccatctttctgaatgtcctggaagccattgagccaggtgtagtgtgtgctggacacgacaacaaccagcccgactcctttgcagccttgctctctagcctcaatgaactgggagagagacagcttgtacacgtggtcaagtgggccaaggccttgcctggcttccgcaacttacacgtggacgaccagatggctgtcattcagtactcctggatggggctcatggtgtttgccatgggctggcgatccttcaccaatgtcaactccaggatgctctacttcgcccctgatctggttttcaatgagtaccgcatgcacaagtcccggatgtacagccagtgtgtccgaatgaggcacctctctcaagagtttggatggctccaaatcaccccccaggaattcctgtgcatgaaagcactgctactcttcagcattattccagtggatgggctgaaaaatcaaaaattctttgatgaacttcgaatgaactacatcaaggaactcgatcgtatcattgcatgcaaaagaaaaaatcccacatcctgctcaagacgcttctaccagctcaccaagctcctggactccgtgcagcctattgcgagagagctgcatcagttcacttttgacctgctaatcaagtcacacatggtgagcgtggactttccggaaatgatggcagagatcatctctgtgcaagtgcccaagatcctttctgggaaagtcaagcccatctatttccacacccagtgaagcattggaaaccctatttccc

SEQ ID NO: 16 - protein, 920 aa, Artificial Sequence
  synthetically generated peptide
MEVQLGLGRVYPRPPSKTYRGAFQNLFQSVREVIQNPGPRHPEAASAAPPGASLLLLQQQQQQQQQQQQQQQQQQQQQQQETSPRQQQQQQGEDGSPQAHRRGPTGYLVLDEEQQPSQPQSALECHPERGCVPEPGAAVAASKGLPQQLPAPPDEDDSAAPSTLSLLGPTFPGLSSCSADLKDILSEASTMQLLQQQQQEAVSEGSSSGRAREASGAPTSSKDNYLGGTSTISDNAKELCKAVSVSMGLGVEALEHLSPGEQLRGDCMYAPLLGVPPAVRPTPCAPLAECKGSLLDDSAGKSTEDTAEYSPFKGGYTKGLEGESLGCSGSAAAGSSGTLELPSTLSLYKSGALDEAAAYQSRDYYNFPLALAGPPPPPPPPHPHARIKLENPLDYGSAWAAAAAQCRYGDLASLHGAGAAGPGSGSPSAAASSSWHTLFTAEEGQLYGPCGGGGGGGGGGGGGGGGGGGGGGGEAGAVAPYGYTRPPQGLAGQESDFTAPDVWYPGGMVSRVPYPSPTCVKSEMGPWMDSYSGPYGDMRLETARDHVLPIDYYFPPQKTCLICGDEASGCHYGALTCGSCKVFFKRAAEGKQKYLCASRNDCTIDKFRRKNCPSCRLRKCYEAGMTLGARKLKKLGNLKLQEEGEASSTTSPTEETTQKLTVSHIEGYECQPIFLNVLEAIEPGVVCAGHDNNQPDSFAALLSSLNELGERQLVHVVKWAKALPGFRNLHVDDQMAVIQYSWMGLMVFAMGWRSFTNVNSRMLYFAPDLVFNEYRMHKSRMYSQCVRMRHLSQEFGWLQITPQEFLCMKALLLFSIIPVDGLKNQKFFDELRMNYIKELDRIIACKRKNPTSCSRRFYQLTKLLDSVQPIARELHQFTFDLLIKSHMVSVDFPEMMAEIISVQVPKILSGKVKPIYFHTQ

SEQ ID NO: 17 - DNA, 2052 bp, Artificial Sequence
  synthetically generated cassette
gaatatatatatattcgtcgagtagctgaatatatatatattcgtcgagtagctggatccgtcgagtagctggtacacagtgttctgtcgagtagctggtacacagtgttctgtcgaggctctagctagtatataaattgataaatcgaaattacagcctaattaatgggacataaaacatatatttatctggcgccagaatttcgacgctctagacggatccgtcgagtagctggtacacagtgttctgtcgagtagctggtacacagtgttctgtcgaggctctagacggtacacagtgttctacctagtcgtcctcgtcttttcttcttcttcttctataaaacaatacccaaagagctggcttgacgaaggcattccggtactgttggtaaagccatggaagacgccaaaaacataaagaaaggcccggcgccattctatccgctggaagatggaaccgctggagagcaactgcataaggctatgaagagatacgccctggttcctggaacaattgcttttacagatgcacatatcgaggtggacatcacttacgctgagtacttcgaaatgtccgttcggttggcagaagctatgaaacgatatgggctgaatacaaatcacagaatcgtcgtatgcagtgaaaactctcttcaattctttatgccggtgttgggcgcgttatttatcggagttgcagttgcgcccgcgaacgacatttataatgaacgtgaattgctcaacagtatgggcatttcgcagcctaccgtggtgttcgtttccaaaaaggggttgcaaaaaattttgaacgtgcaaaaaaagctcccaatcatccaaaaaattattatcatggattctaaaacggattaccagggatttcagtcgatgtacacgttcgtcacatctcatctacctcccggttttaatgaatacgattttgtgccagagtccttcgatagggacaagacaattgcactgatcatgaactcctctggatctactggtctgcctaaaggtgtcgctctgcctcatagaactgcctgcgtgagattctcgcatgccagagatcctatttttggcaatcaaatcattccggatactgcgattttaagtgttgttccattccatcacggttttggaatgtttactacactcggatatttgatatgtggatttcgagtcgtcttaatgtatagatttgaagaagagctgtttctgaggagccttcaggattacaagattcaaagtgcgctgctggtgccaaccctattctccttcttcgccaaaagcactctgattgacaaatacgatttatctaatttacacgaaattgcttctggtggcgctcccctctctaaggaagtcggggaagcggttgccaagaggttccatctgccaggtatcaggcaaggatatgggctcactgagactacatcagctattctgattacacccgagggggatgataaaccgggcgcggtcggtaaagttgttccattttttgaagcgaaggttgtggatctggataccgggaaaacgctgggcgttaatcaaagaggcgaactgtgtgtgagaggtcctatgattatgtccggttatgtaaacaatccggaagcgaccaacgccttgattgacaaggatggatggctacattctggagacatagcttactgggacgaagacgaacacttcttcatcgttgaccgcctgaagtctctgattaagtacaaaggctatcaggtggctcccgctgaattggaatccatcttgctccaacaccccaacatcttcgacgcaggtgtcgcaggtcttcccgacgatgacgccggtgaacttcccgccgccgttgttgttttggagcacggaaagacgatgacggaaaaagagatcgtggattacgtcgccagtcaagtaacaaccgcgaaaaagttgcgcggaggagttgtgtttgtggacgaagtaccgaaaggtcttaccggaaaactcgacgcaagaaaaatcagagagatcctcataaaggccaagaagggcggaaagatcgccgtgtaa

SEQ ID NO: 18 - protein, 550 aa, Artificial Sequence; identical to SEQ ID NO: 10
  synthetically generated peptide

SEQ ID NO: 19 - DNA, 15 bp, Artificial Sequence
  synthetic repeat
agaacactgtgtacc

SEQ ID NO: 20 - protein, 48 aa, Artificial Sequence
  synthetically generated peptide
HMINWAKRVPGFVDLTLHDQVHLLECAWLEILMIGLVWRSMEHPVKLL

SEQ ID NO: 21 - protein, 44 aa, Artificial Sequence
  synthetically generated peptide
VLVEFTKKLPGFQTLDHEDQIALLKGSAVEAMFLRSAEIFNKKL

SEQ ID NO: 22 - protein, 48 aa, Artificial Sequence
  synthetically generated peptide
EIVDFAKQLPGFLQLSREDQIALLKTSAIEVMLLETSRRYNPGSESIT

SEQ ID NO: 23 - protein, 48 aa, Artificial Sequence
  synthetically generated peptide
ELTEFAKAIPGFANLDLNDQVTLLKYGVYEAIFAMLSSVMNKDGMLVA

SEQ ID NO: 24 - protein, 48 aa, Artificial Sequence
  synthetically generated peptide
EITEYAKSIPGFVNLDLNDQVTLLKYGVHEIIYTMLASLMNKDGVLIS

SEQ ID NO: 25 - protein, 48 aa, Artificial Sequence
  synthetically generated peptide
KTVEFAKQLPGFTTLTIADQITLLKAACLDILILRICTRYTPEQDTMT

SEQ ID NO: 26 - protein, 48 aa, Artificial Sequence
  synthetically generated peptide
TLVEWAKRIPHFSELPLDDQVILLRAGWNELLIASFSHRSIAVKDGIL

SEQ ID NO: 27 - protein, 48 aa, Artificial Sequence
RVVDFAKKLPMFSELPCEDQIILLKGCCMEIMSLRAAVRYDPESDTLT

SEQ ID NO: 28 - protein, 70 aa, Artificial Sequence
  synthetically generated peptide
TRYCAVCNDYASGYHYGVWSCEGCKAFFKRSIQGHNDYMCPATNQCTIDKNRRKSCQACRLRKCYEVGMM

SEQ ID NO: 29 - protein, 70 aa, Artificial Sequence
  synthetically generated peptide
DELCVVCGDRASGYHYNALTCEGCKGFFRRSITKNAVYKCKNGGNCVMDMYMRRKCQECRLRKCKEMGML

SEQ ID NO: 30 - protein, 70 aa, Artificial Sequence
  synthetically generated peptide
NELCSVCGDKASGFHYNVLSCEGCKGFFRRSVIKGAHYICHSGGHCPMDTYMRRKCQECRLRKCRQAGMR

SEQ ID NO: 31 - protein, 69 aa, Artificial Sequence
  synthetically generated peptide
NIECRICGDKASGYHYGVHACEGCKGFFRRTIRLKLVYDKCDRSCKIQKKNRNKCQYCRFHKCLSVGMS

SEQ ID NO: 32 - protein, 69 aa, Artificial Sequence
  synthetically generated peptide
AIECRVCGDKASGFHYGVHACEGCKGFFRRTIRLKLIYDRCDLNCRIHKKSRNKCQYCRFQKCLAVGMS

SEQ ID NO: 33 - protein, 70 aa, Artificial Sequence
  synthetically generated peptide
YKPCFVCQDKSSGYHYGVSACEGCKGFFRRSIQKNMVYTCHRDKNCIINKVTRNRCQYCRLQKCFEVGMS

SEQ ID NO: 34 - protein, 70 aa, Artificial Sequence
  synthetically generated peptide
KHICAICGDRSSGKHYGVYSCEGCKGFFKRTVRKDLTYTCRDNKDCLIDKRQRNRCQYCRYQKCLAMGMK

SEQ ID NO: 35 - protein, 72 aa, Artificial Sequence
  synthetically generated peptide
DEQCVVCGDKATGYHYRCITCEGCKGFFRRTIQKNLHPTYSCKYDSCCVIDKITRNQCQLCRFKKCIAVGMA

SEQ ID NO: 36 - protein, 8 aa, Artificial Sequence
  synthetically generated tag peptide
DYKDDDDK

SEQ ID NO: 37 - protein, 9 aa, Artificial Sequence
  synthetically generated tag peptide
YPYDVPDYA

SEQ ID NO: 38 - protein, 10 aa, Artificial Sequence
  synthetically generated epitope peptide
EQKLISEEDL

SEQ ID NO: 39 - protein, 6 aa, Artificial Sequence
  synthetically generated tag peptide
DTYRYI

SEQ ID NO: 40 - protein, 6 aa, Artificial Sequence
  synthetically generated tag peptide
HHHHHH

SEQ ID NO: 41 - DNA, 15 bp, Artificial Sequence
  synthetically generated repeat
agaacannntgttct

SEQ ID NO: 42 - DNA, 15 bp, Artificial Sequence
  synthetically generated repeat
aggtcannntgacct

SEQ ID NO: 43 - DNA, 13 bp, Artificial Sequence
  synthetically generated repeat
aggtcanaggtca

SEQ ID NO: 44 - DNA, 14 bp, Artificial Sequence
  synthetically generated repeat
aggtcannaggtca

SEQ ID NO: 45 - DNA, 15 bp, Artificial Sequence
  synthetically generated repeat
aggtcannnaggtca

SEQ ID NO: 46 - DNA, 16 bp, Artificial Sequence
  synthetically generated repeat
aggtcannnnaggtca

SEQ ID NO: 47 - DNA, 17 bp, Artificial Sequence
  synthetically generated repeat
aggtcannnnnaggtca

SEQ ID NO: 48 - protein, 4 aa, Artificial Sequence
  synthetically generated peptide
HDEL